(19) United States
(12) Patent Application Publication
(10) Pub. No.: US 2010/0167986 A1
(43) Pub. Date: Jul. 1, 2010

(54) Cholesteryl Ester Transfer Protein Inhibitors

(76) Inventors: Amjad Ali, Freehold, NJ (US); Peter J. Sinclair, Scotch Plains, NJ (US)

Correspondence Address: MERCK, P O BOX 2000, RAHWAY, NJ, 07065-0907, US

(21) Appl. No.: 11/992,816

(22) PCT Filed: Sep. 28, 2006
(86) PCT No.: PCT/US06/38435

§ 371 Date: Mar. 27, 2008

Related U.S. Application Data
(60) Provisional application No. 60/722,229, filed on Sep. 30, 2005.

(51) Int. Cl. A61K 38/28; C07D 257/00; A61K 31/41; C07D 249/04; A61K 31/4192; C07D 261/14; A61K 31/42; A61P 9/10; A61K 38/16; A61P 3/04; A61P 9/12

(52) U.S. Cl. 514/4; 548/251; 514/381; 548/255; 514/359; 548/246; 514/380; 514/12

(57) ABSTRACT

Compounds of Formula (I), including pharmaceutically acceptable salts of the compounds, are CETP inhibitors, and are useful for raising HDL-cholesterol, reducing LDL-cholesterol, and for treating or preventing atherosclerosis. In the compounds of Formula (I), A¹ and A² are each an aromatic ring, a 5-6-membered heterocyclic ring, an aromatic ring fused to a heterocyclic ring, a phenyl ring fused to a heterocyclic ring, or a cycloalkyl ring, and Z is an aromatic or heterocyclic ring.

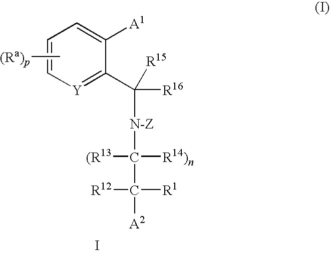

### FIELD OF THE INVENTION

This invention relates to a class of chemical compounds that inhibit cholesteryl ester transfer protein (CETP) and therefore may have utility in the treatment and prevention of atherosclerosis.

### BACKGROUND OF THE INVENTION

Atherosclerosis and its clinical consequences, coronary heart disease (CHD), stroke and peripheral vascular disease, represent a truly enormous burden to the health care systems of the industrialized world. In the United States alone, approximately 13 million patients have been diagnosed with CHD, and greater than one half million deaths are attributed to CHD each year. Further, this toll is expected to grow over the next quarter century as an epidemic in obesity and diabetes continues to grow.

It has long been recognized that in mammals, variations in circulating lipoprotein profiles correlate with the risk of atherosclerosis and CHD. The clinical success of HMG-CoA Reductase inhibitors, especially the statins, in reducing coronary events is based on the reduction of circulating Low Density Lipoprotein cholesterol (LDL-C), levels of which correlate directly with increased risk for atherosclerosis. More recently, epidemiologic studies have demonstrated an inverse relationship between High Density Lipoprotein cholesterol (HDL-C) levels and atherosclerosis, leading to the conclusion that low serum HDL-C levels are associated with an increased risk for CHD.

Metabolic control of lipoprotein levels is a complex and dynamic process involving many factors. One important metabolic control in man is the cholesteryl ester transfer protein (CETP), a plasma glycoprotein that catalyzes the movement of cholesteryl esters from HDL to the apoB containing lipoproteins, especially VLDL (see Hesler, C. B., et. al. (1987) Purification and characterization of human plasma cholesteryl ester transfer protein. J. Biol. Chem. 262(5), 2275-2282)). Under physiological conditions, the net reaction is a heteroexchange in which CETP carries triglyceride to HDL from the apoB lipoproteins and transports cholesterol ester from HDL to the apoBliprotein.

In humans, CETP plays a role in reverse cholesterol transport, the process whereby cholesterol is returned to the liver from peripheral tissues. Intriguingly, many animals do not possess CETP, including animals that have high HDL levels and are known to be resistant to coronary heart disease, such as rodents (see Guyard-Dangremont, V., et. al., (1998) Phospholipid and cholesteryl ester transfer activities in plasma from 14 vertebrate species. Relation to atherogenesis susceptibility, Comp. Biochem. Physiol. B Biochem. Mol. Biol. 120(3), 517-525). Numerous epidemiologic studies correlating the effects of natural variation in CETP activity with respect to coronary heart disease risk have been performed, including studies on a small number of known human null mutations (see Hirano, K.-I., Yamashita, S. and Matsuzawa, Y. (2000) Pros and cons of inhibiting cholesteryl ester transfer protein, Curr. Opin. Lipidol. 11(6), 589-596). These studies have clearly demonstrated an inverse correlation between plasma HDL-C concentration and CETP activity (see Inazu, A., et. al. (2000) Cholesteryl ester transfer protein and atherosclerosis, Curr. Opin. Lipidol. 11(4), 389-396), leading to the hypothesis that pharmacologic inhibition of CETP lipid transfer activity may be beneficial to humans by increasing levels of HDL-C while lowering those of LDL.

Despite the significant therapeutic advance that statins such as simvastatin (ZOCOR®) represent, statins only achieve a risk reduction of approximately one-third in the treatment and prevention of atherosclerosis and ensuing atherosclerotic disease events. Currently, few pharmacologic therapies are available that favorably raise circulating levels of HDL-C. Certain statins and some fibrates offer modest HDL-C gains. Niacin, which provides the most effective therapy for raising HDL-C that has been clinically documented, suffers from patient compliance issues, due in part to side effects such as flushing. An agent that safely and effectively raises HDL cholesterol levels can answer a significant, but as yet unmet medical need by offering a means of pharmacologic therapy that can significantly improve circulating lipid profiles through a mechanism that is complementary to existing therapies.

New classes of chemical compounds that inhibit CETP are being investigated at several pharmaceutical companies or are in clinical trials. No CETP inhibitors are currently being marketed. New compounds are needed so that one or more pharmaceutical compounds can be found that are safe and effective. The novel compounds described herein are very potent CETP inhibitors. Some structurally similar compounds are found in WO2005/100298 and WO2006/056854, both of which published after the priority date of this application.

### SUMMARY OF THE INVENTION

Compounds having Formula I, including pharmaceutically acceptable salts of the compounds, are CETP inhibitors, having the utilities described below:

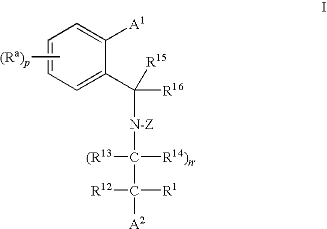

The phenyl ring of Formula I may optionally have —N═ in place of —(CH)═ at one of the 4 positions that does not have a substituent group in Figure I.

In the compound having formula I,

A¹ is selected from the group consisting of:
(a) an aromatic ring selected from phenyl and naphthyl;
(b) a phenyl ring fused to a 5-7 membered non-aromatic cycloalkyl ring, which optionally comprises 1-2 double bonds;
(c) a 5-6-membered heterocyclic ring having 1-4 heteroatoms independently selected from N, S, and O, and optionally also comprising 1-3 double bonds and a carbonyl group or —N(O)— group, wherein the point of attachment of A¹ to the phenyl ring to which A¹ is attached is a carbon atom; and
(d) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A¹ to the phenyl ring to which A¹ is attached is a carbon atom of A¹;

wherein A¹ is optionally substituted with 1-5 substituent groups independently selected from R^{b};

A² is selected from the group consisting of:
(a) an aromatic ring selected from phenyl and naphthyl;
(b) a phenyl ring fused to a 5-7 membered non-aromatic cycloalkyl ring, which optionally comprises 1-2 double bonds;
(c) a 5-6-membered heterocyclic ring having 1-4 heteroatoms independently selected from N, S, and O, and optionally also comprising 1-3 double bonds and a carbonyl group or —N(O)— group, wherein the point of attachment of A² is a carbon atom of A²;
(d) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A² is a carbon atom of A²; and
(e) a —C₃-C₈ cycloalkyl ring optionally having 1-3 double bonds;

wherein A² is optionally substituted with 1-5 substituent groups independently selected from R^{c};

Each R^{a} and R^{c} is independently selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆alkyl, —OC₂-C₆ alkenyl, —OC₂-C₆ alkynyl, —OC₃-C₈ cycloalkyl optionally having 1-3 double bonds, —C(═O)C₁-C₆alkyl, —C(═O)C₃-C₈ cycloalkyl, —C(═O)H, —CO₂H, —CO₂C₁-C₆alkyl, —C(═O)SC₁-C₆alkyl, —NR¹⁰R¹¹, —C(═O)NR¹⁰R¹¹, —NR¹⁰C(═O)OC₁-C₆ alkyl, —NR¹⁰C(═O)NR¹⁰R¹¹, —S(O)ₓC₁-C₆ alkyl, —S(O)_{y}NR¹⁰R¹¹, —NR¹⁰S(O)_{y}NR¹⁰R¹¹, halogen, —CN, —NO₂, and a 5-6-membered heterocyclic ring having 1-4 heteroatoms independently selected from N, S, and O, said heterocyclic ring optionally also comprising a carbonyl group and optionally also comprising 1-3 double bonds,

wherein when R^{a} and R^{c} are selected from the group consisting of a heterocyclic ring, —C₃-C₈ cycloalkyl, —OC₃-C₈ cycloalkyl, and —C(═O)C₃-C₈ cycloalkyl, the heterocyclic ring and —C₃-C₈ cycloalkyl groups of R^{a} and R^{c} are optionally substituted with 1-5 substituent groups independently selected from halogen, —C₁-C₃ alkyl, and —OC₁-C₃ alkyl, wherein —C₁-C₃ alkyl and —OC₁-C₃ alkyl are optionally substituted with 1-7 halogens,

when R^{a} and R^{c} are selected from the group consisting of —C₁-C₆ alkyl, —C₂-C₆ alkenyl, —C₂-C₆ alkynyl, —OC₁-C₆alkyl, —OC₂-C₆ alkenyl, —OC₂-C₆ alkynyl, —C(═O)C₁-C₆alkyl, —CO₂C₁-C₆alkyl, —C(═O)SC₁-C₆alkyl, —NR¹⁰C(═O)OC₁-C₆ alkyl, and —S(O)ₓC₁-C₆ alkyl, the alkyl, alkenyl, and alkynyl groups of R^{a} and R^{c} are optionally substituted with 1-13 halogens and are optionally substituted with 1-3 substituent groups independently selected from (a) —OH, (b) —CN, (c) —NR¹⁰R¹¹, (d) —C₃-C₈ cycloalkyl optionally having 1-3 double bonds and optionally substituted with 1-15 halogens, (e) —OC₁-C₄alkyl optionally substituted with 1-9 halogens and optionally also substituted with 1-2 substituent groups independently selected from —OC₁-C₂ alkyl, (f) —OC₃-C₈ cycloalkyl optionally having 1-3 double bonds and optionally substituted with 1-15 halogens, (g) —CO₂H, (h) —C(═O)CH₃, and (i) —CO₂C₁-C₄alkyl which is optionally substituted with 1-9 halogens;

wherein 2 groups R^{a} that are on adjacent carbon atoms of the phenyl or optional pyridinyl ring of Formula I may optionally be joined to form a bridging moiety selected from —CH₂CH₂CH₂—, —CH₂CH₂CH₂CH₂—, and —CH═CH—CH═CH—, thereby yielding a cyclopentyl, cyclohexyl, or phenyl ring fused to the phenyl ring or optional pyridinyl ring of Formula I, wherein said cyclopentyl, cyclohexyl, or phenyl ring that is fused to the phenyl or optional pyridinyl ring of Formula I is optionally substituted with 1-2 groups R^{a};

Each R^{b} is independently selected from the group consisting of —C₁-C₆ alkyl, —C₂-C₆ alkenyl, —C₂-C₆ alkynyl, —C₃-C₈ cycloalkyl optionally having 1-3 double bonds, —OC₁-C₆alkyl, —OC₂-C₆ alkenyl, —OC₂-C₆ alkynyl, —OC₃-C₈ cycloalkyl optionally having 1-3 double bonds, —C(═O)C₁-C₆alkyl, —C(═O)C₃-C₈ cycloalkyl, —C(═O)H, —CO₂H, —CO₂C₁-C₆alkyl, —C(═O)SC₁-C₆alkyl, —NR¹⁰R¹¹, —C(═O)NR¹⁰R¹¹, —NR¹⁰C(═O)OC₁-C₆ alkyl, —NR¹⁰C(═O)NR¹⁰R¹¹, —S(O)ₓC₁-C₆ alkyl, —S(O)_{y}NR¹⁰R¹¹, —NR¹⁰S(O)_{y}NR¹⁰R¹¹, halogen, —CN, —NO₂, phenyl, and a 5-6-membered heterocyclic ring having 1-4 heteroatoms independently selected from N, S, and O, said heterocyclic ring optionally also comprising a carbonyl group and optionally also comprising 1-3 double bonds,

wherein when R^{b} is selected from the group consisting of a heterocyclic ring, —C₃-C₈ cycloalkyl, —OC₃-C₈ cycloalkyl, and —C(═O)C₃-C₈ cycloalkyl, the heterocyclic ring and —C₃-C₈ cycloalkyl groups of R^{b} are optionally substituted with 1-5 substituent groups independently selected from halogen, —C₁-C₃ alkyl, —C₂-C₃ alkenyl, —NR¹⁰R¹¹, —OC₁-C₃ alkyl, —CO₂H, —CN, and —CO₂C₁-C₃alkyl, wherein —C₁-C₃ alkyl and —C₂-C₃ alkenyl in all uses are optionally substituted with 1-7 halogens and optionally one group —OH,

when R^{b} is selected from the group consisting of —C₁-C₆ alkyl, —C₂-C₆ alkenyl, —C₂-C₆ alkynyl, —OC₁-C₆alkyl, —OC₂-C₆ alkenyl, —OC₂-C₆ alkynyl, —C(═O)C₁-C₆alkyl, —CO₂C₁-C₆alkyl, —C(═O)SC₁-C₆alkyl, —NR¹⁰C(═O)OC₁-C₆ alkyl, and —S(O)ₓC₁-C₆ alkyl, the alkyl, alkenyl, and alkynyl groups of R^{b} are optionally substituted with 1-13 halogens and are optionally substituted with 1-3 substituent groups independently selected from (a) —OH, (b) —CN, (c) —NR¹⁰R¹¹, (d) —C₃-C₈ cycloalkyl optionally having 1-3 double bonds and optionally substituted with 1-15 halogens, (e) —OC₁-C₄alkyl optionally substituted with 1-9 halogens and optionally also substituted with 1-2 substituent groups independently selected from —OC₁-C₂ alkyl, (f) —OC₃-C₈ cycloalkyl optionally having 1-3 double bonds and optionally substituted with 1-15 halogens, (g) —CO₂H, (h) —C(═O)CH₃, and (i) —CO₂C₁-C₄alkyl which is optionally substituted with 1-9 halogens;

and when R^{b} is phenyl, said phenyl is optionally substituted with 1-5 halogens and is optionally substituted with 1-3 substituents independently selected from —C₁-C₄ alkyl, —C₂-C₄ alkenyl, —C₂-C₄ alkynyl, —C₃-C₆ cycloalkyl, —OC1-C₄ alkyl, —OC₂-C₄ alkenyl, —OC₂-C₄ alkynyl, —OC₃-C₆ cycloalkyl, —C(═O)C₁-C₄alkyl, —C(═O)H, —CO₂H, —CO₂C₁-C₄alkyl, —NR¹⁰R¹¹, —C(═O)NR¹⁰R¹¹, —NR¹⁰C(═O)OC₁-C₄ alkyl, —NR¹⁰C(═O)NR¹⁰R¹¹, —S(O)ₓC₁-C₄ alkyl, —S(O)_{y}NR¹⁰R¹¹, —NR¹⁰S(O)_{y}NR¹⁰R¹¹, —CN, —NO₂, and a 5-6-membered heterocyclic ring having 1-4 heteroatoms independently selected from N, S, and O, said heterocyclic ring optionally also comprising a carbonyl group and optionally also comprising 1-3 double bonds and optionally comprising 1-3 substituents independently selected from halogen, —CH₃, —OCH₃, —CF₃, and —OCF₃; wherein when the substituents on phenyl when Rb is phenyl are selected from —C₁-C₄ alkyl, —C₂-C₄ alkenyl, —C₂-C₄ alkynyl, —C₃-C₆ cycloalkyl, —OC₁-C₄alkyl, —OC₂-C₄ alkenyl, —OC₂-C₄ alkynyl, —OC₃-C₆ cycloalkyl, —C(═O)C₁-C₄alkyl, —CO₂C₁-C₄alkyl, —NR¹⁰C(═O)OC₁-C₄ alkyl, and —S(O)ₓC₁-C₄ alkyl, then the alkyl, alkenyl, alkynyl, and cycloalkyl groups of said substituent groups optionally comprise 1-5 halogen substituents and optionally comprise one substituent selected from —OH, —NR¹⁰R¹¹, —OCH₃ optionally substituted with 1-3 F, and phenyl which is optionally substituted with 1-3 substituents independently selected from halogen, —CH₃, —OCH₃, —CF₃, and —OCF₃;

n is an integer selected from 0 and 1;

p is an integer from 0-4;

x is an integer selected from 0, 1, and 2;

y is an integer selected from 1 and 2;

Z is phenyl or a 5-6-membered heterocyclic ring having 1-4 heteroatoms independently selected from N, S, and O, said heterocyclic ring optionally also comprising a carbonyl group and 1-3 double bonds, said heterocyclic ring being connected by a carbon atom to the N to which said heterocyclic ring is attached, wherein said phenyl or 5-6-membered heterocyclic ring optionally comprises 1-3 substituents independently selected from -halogen, C₁-C₄ alkyl, —C₂-C₄ alkenyl, —C₂-C₄ alkynyl, —OC₁-C₄alkyl, —OC₂-C₄ alkenyl, —OC₂-C₄ alkynyl, —C(═O)C₁-C₄alkyl, CO₂C₁-C₄alkyl, —NR¹⁰R¹¹, —C(═O)NR¹⁰R¹¹, —NR¹⁰C(═O)OC₁-C₄ alkyl, —NR¹⁰C(═O)NR¹⁰R¹¹, —S(O)ₓC₁-C₄ alkyl, —S(O)_{y}NR¹⁰R¹¹, —NR¹⁰S(O)_{y}NR¹⁰R¹¹, —OH, —CN, and —NO₂, wherein the alkyl, alkenyl, and alkynyl groups of said substituents are optionally substituted with 1-5 halogens and optionally one substituent selected from —OH and —CO₂H;

R¹, R¹², R¹³, R¹⁴, R¹⁵, and R¹⁶ are each independently selected from the group consisting of H, —OH, halogen, —C₁-C₄ alkyl, —C₃-C₆ cycloalkyl, —OC₁-C₄ alkyl, and —NR¹⁰R¹¹, wherein —C₁-C₄ alkyl, —C₃-C₆ cycloalkyl, and —OC₁-C₄ alkyl are each optionally substituted with 1-9 halogens and are each optionally also substituted with 1-2 groups independently selected from —OH, —C(═O)CH₃, —OC(═O)CH₃, —OC₁-C₂ alkyl, and —OC₁-C₂ alkyleneOC₁-C₂alkyl, wherein R¹ and R¹² together may optionally form an oxo group; and

R¹⁰ and R¹¹ are each independently selected from H, —C₁-C₅ alkyl, —C(═O)C₁-C₅ alkyl and —S(O)_{y}C₁-C₅ alkyl, wherein —C₁-C₅ alkyl in all instances is optionally substituted with 1-11 halogens.

In the compounds of Formula I and in subsequent compounds, alkyl, alkenyl, and alkynyl groups can be either linear or branched, unless otherwise stated.

### DETAILED DESCRIPTION OF THE INVENTION

In a subset of the compound of Formula I, the various groups have the following definitions:

In a subset of compounds,
A¹ is selected from the group consisting of
(a) an aromatic ring selected from phenyl and naphthyl;
(b) a 5-6-membered heterocyclic ring having 1-4 heteroatoms independently selected from N, S, and O, and optionally also comprising 1-3 double bonds and a carbonyl group or —N(O)— group, wherein the point of attachment of A¹ to the phenyl ring to which A¹ is attached is a carbon atom; and
(c) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A¹ to the phenyl ring to which A¹ is attached is a carbon atom;

wherein A¹ is optionally substituted with 1-4 substituent groups independently selected from —C₁-C₅ alkyl, —OC₁-C₃alkyl, —CO₂C₁-C₃alkyl, —CO₂H, halogen, —NR¹⁰R¹¹, —C(═O)C₁-C₃ alkyl, —C(═O)H, —S(O)ₓC₁-C₃ alkyl, —C₂-C₃ alkenyl, —CN, —NO₂, —C₃-C₆ cycloalkyl, phenyl, and a 5-6-membered heterocyclic ring having 1-3 heteroatoms independently selected from N, S, and O, and optionally also comprising 1-3 double bonds, wherein —C₁-C₃ alkyl, —C₁-C₅ alkyl, and —C₂-C₃ alkenyl in all occurrences are optionally substituted with 1-6 substituents independently selected from 1-5 halogens and one —OH or —CO₂H group; and —C₃-C₆ cycloalkyl, phenyl, and 5-6-membered heterocyclic ring are optionally substituted with 1-3 substituents independently selected from halogen, —C₁-C₃ alkyl, —C₂-C₃ alkenyl, —OC₁-C₃ alkyl, —NR¹⁰R¹¹, —CO₂H, —CO₂C₁-C₃ alkyl, and —CN, wherein —C₁-C₃ alkyl and —C₂-C₃ alkenyl in all uses are optionally substituted with 1-3 halogens and optionally one —OH group.

In a subset of compounds, A² is selected from the group consisting of phenyl, naphthyl, —C₃-C₆ cycloalkyl, and a heterocyclic 5-6 membered ring having 1-3 heteroatoms independently selected from O, N, and S, and optionally also comprising 1-3 double bonds and a carbonyl group or —N(O)— group, wherein A² is optionally substituted with 1-2 substituent groups independently selected from —C₁-C₄ alkyl, —OC₁-C₃ alkyl, —C(═O)C₁-C₃alkyl, —C(═O)H, —NO₂, —CN, —S(O)ₓC₁-C₃ alkyl, —NHS(O)₂C₁-C₃ alkyl, —NR¹⁰R¹¹, —NR¹⁰C(═O)R¹¹, —C₂-C₃ alkenyl, —C(═O)NR¹⁰R¹¹, halogen, —C₃-C₆ cycloalkyl, and a 5-6-membered heterocyclic ring having 1-3 heteroatoms independently selected from N, S, and O, and optionally also comprising 1-3 double bonds, wherein C₁-C₃ alkyl, C₁-C₄ alkyl, and C₂-C₃alkenyl in all instances are optionally substituted with 1-3 halogens, and —C₃-C₆ cycloalkyl and the 5-6-membered heterocyclic ring are optionally substituted with 1-3 substituents independently selected from halogen and —C₁-C₃ alkyl.

In a subset of compounds, each R^{a} is independently selected from the group consisting of H, halogen, —NR¹⁰R¹¹, —C₁-C₃ alkyl, —OC₁-C₃ alkyl, —C₂-C₃ alkenyl, —C₃-C₆ cycloalkyl optionally having a double bond, —OC₃-C₆ cycloalkyl optionally having a double bond, —C(═O)C₁-C₃alkyl, —C(═O)C₃-C₆ cycloalkyl, —C(═O)H, —CO₂H, —CO₂C₁-C₃alkyl, —C(═O)NR¹⁰R¹¹, —CN, —NO₂, and a 5-6-membered heterocyclic ring having 1-4 heteroatoms independently selected from N, S, and O, and optionally 1-3 double bonds, wherein C₁-C₃ alkyl and —C₂-C₃ alkenyl in all instances are optionally substituted with 1-5 halogens, and —C₃-C₆ cycloalkyl and the 5-6-membered heterocyclic ring are in all occurrences optionally substituted with 1-3 substituents independently selected from halogen, —C₁-C₃ alkyl, —OC₁-C₃ alkyl, —CF₃, and —OCF₃;

wherein 2 groups R^{a} that are on adjacent carbon atoms of the phenyl or optional pyridinyl ring of Formula I may optionally be joined to form a bridging moiety selected from —CH₂CH₂CH₂, —CH₂CH₂CH₂CH₂—, and —CH═CH—CH═CH—, thereby yielding a cyclopentyl, cyclohexyl, or phenyl ring fused to the phenyl ring or optional pyridinyl ring of Formula I, wherein said cyclopentyl, cyclohexyl, or phenyl ring that is fused to the phenyl or optional pyridinyl ring of Formula I is optionally substituted with 1-2 groups R^{a}.

In subset of compounds, R¹ is selected from the group consisting of H, F, OH, C₁-C₃ alkyl, and —OC₁-C₃ alkyl, wherein C₁-C₃ alkyl and —OC₁-C₃ alkyl are each optionally substituted with 1-3 halogens and also are optionally substituted with one —OC₁-C₂alkyl.

In a subset of compounds, R¹⁰ and R¹¹ are each independently selected from H and —C₁-C₃ alkyl.

In a subset of compounds, R¹², R¹³, R¹⁴, R¹⁵, and R¹⁶ are each H.

In a subset of compounds, Z is selected from phenyl and a 5-6-membered heterocyclic ring having 1-4 heteroatoms independently selected from N, S, and O, said heterocyclic ring optionally also comprising 1-3 double bonds, said heterocyclic ring being connected by a carbon atom to the N to which said heterocyclic ring is attached, wherein said phenyl or 5-6-membered heterocyclic ring optionally comprises 1-3 substituents independently selected from halogen, C₁-C₄ alkyl, —C₂-C₄ alkenyl, —C₂-C₄ alkynyl, —OC₁-C₄alkyl, —OC₂-C₄ alkenyl, —OC₂-C₄ alkynyl, —C(═O)C₁-C₄alkyl, —CO₂C₁-C₄alkyl, —NR¹⁰R¹¹, —OH, —CN, and —NO₂, wherein the alkyl, alkenyl, and alkynyl groups of said substituents are optionally substituted with 1-5 halogens and optionally one substituent selected from —OH, —CO₂H, and —CO₂C₁-C₄alkyl.

Many of compounds of this invention have a structure in accordance with Formula Ia, written below, or a pharmaceutically acceptable salt thereof:

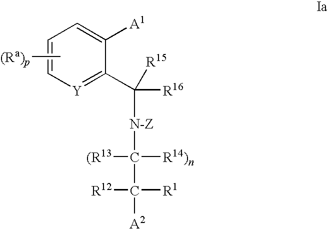

A subset of the compounds has Formula Ib, including pharmaceutically acceptable salts thereof:

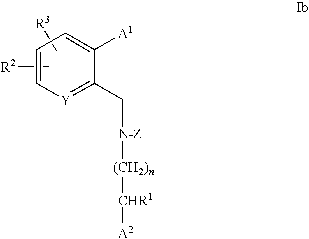

Another subset of compounds has Formula Ic, including pharmaceutically acceptable salts thereof:

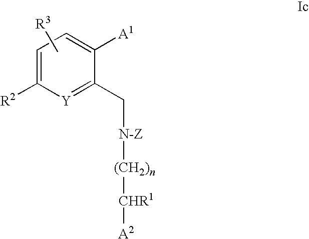

In Formula Ia, Ib and Ic, Y is selected from the group consisting of —N═ and —CH═.

In Formula Ib, R² and R³ are each independently selected from the group consisting of H, halogen, —NR¹⁰R¹¹, —C₁-C₃ alkyl, —OC₁-C₃ alkyl, —C₂-C₃ alkenyl, —C₃-C₆ cycloalkyl optionally having a double bond, —OC₃-C₆ cycloalkyl optionally having a double bond, —C(═O)C₁-C₃alkyl, —C(═O)C₃-C₆ cycloalkyl, —C(═O)H, —CO₂H, —CO₂C₁-C₃alkyl, —C(═O)NR¹⁰R¹¹, —CN, —NO₂, and a 5-6-membered heterocyclic ring having 1-4 heteroatoms independently selected from N, S, and O, and optionally 1-3 double bonds, wherein C₁-C₃ alkyl and —C₂-C₃ alkenyl in all instances are optionally substituted with 1-5 halogens, and —C₃-C₆ cycloalkyl and the 5-6-membered heterocyclic ring are in all occurrences optionally substituted with 1-3 substituents independently selected from halogen, —C₁-C₃ alkyl, —OC₁-C₃ alkyl, —CF₃, and —OCF₃;
wherein if R² and R³ are on adjacent carbon atoms of the phenyl or optional pyridinyl ring of Formula Ib, then R² and R³ may optionally be joined to form a bridging moiety selected from —CH₂CH₂CH₂—, —CH₂CH₂CH₂CH₂—, and —CH═CH—CH═CH—, thereby yielding a cyclopentyl, cyclohexyl, or phenyl ring fused to the phenyl ring or optional pyridinyl ring of Formula I, wherein said cyclopentyl, cyclohexyl, or phenyl ring that is fused to the phenyl or optional pyridinyl ring of Formula Ib is optionally substituted with 1-2 groups R^{a}, and the remaining 1 or 2 —CH═ positions of the phenyl or optional pyridinyl of figure Ib are optionally substituted with R^{a}.

Other groups may be defined as described previously.

In embodiments of the compounds of Formula Ib, R², R³, and R^{a} are each independently selected from the group consisting of H, halogen, —NR¹⁰R¹¹, —C₁-C₃ alkyl, —C₂-C₃ alkenyl, —OC₁-C₃ alkyl, —CN, —NO₂, and pyridyl, wherein C₁-C₃ alkyl and —C₂-C₃ alkenyl in all instances are optionally substituted with 1-3 halogens, or a pharmaceutically acceptable salt thereof.

In the compounds of Ic, R² is selected from the group consisting of H, halogen, cyclopropyl, —NR¹⁰R¹¹, —C₁-C₃ alkyl, —C₂-C₃ alkenyl, —OC₁-C₃ alkyl, —CN, —NO₂, and pyridyl, wherein cyclopropyl, C₁-C₃ alkyl and C₂-C₃ alkenyl in all instances are optionally substituted with 1-3 halogens, and pyridyl is optionally substituted with 1-3 substituents independently selected from the group consisting of halogen, —CH₃, —CF₃, —OCH₃, and —OCF₃;

R³ is selected from the group consisting of H, halogen, —CH₃, —CF₃, —OCH₃, and —OCF₃;

and R^{a} is selected from the group consisting of halogen, —NR¹⁰R¹¹, —C₁-C₃ alkyl, —C₂-C₃ alkenyl, —OC₁-C₃ alkyl, —CN, —NO₂, and pyridinyl, wherein C₁-C₃ alkyl and C₂-C₃ alkenyl in all instances is optionally substituted with 1-3 halogens, and pyridinyl is optionally substituted with 1-3 substituents independently selected from the group consisting of halogen, —CH₃, —CF₃, —OCH₃, and —OCF₃, or a pharmaceutically acceptable salt thereof.

In embodiments of the compounds described above, A¹ is selected from the group consisting of phenyl, thienyl, furyl, pyridyl, 1-oxidopyridinyl, quinolyl, isoquinolyl, benzofuranyl, dihydrobenzofuranyl, indolyl, dihydroindolyl, oxazolyl, isoxazolyl, and oxadiazolyl. A¹ is optionally substituted as described previously.

In other embodiments, A¹ is selected from the group consisting of phenyl, thienyl, furyl, pyridyl, quinolyl, isoquinolyl, benzofuranyl, dihydrobenzofuranyl, indolyl, dihydroindolyl, oxazolyl, and isoxazolyl. In many preferred embodiments, A¹ is phenyl. In either case, A¹ is optionally substituted as described previously.

In embodiments of the compounds described above, A² is selected from the group consisting of phenyl, thienyl, furyl, pyridyl, 1-oxidopyridinyl, quinolyl, isoquinolyl, benzofuranyl, dihydrobenzofuranyl, indolyl, dihydroindolyl, oxazolyl, isoxazolyl, oxadiazolyl, and C₃-C₆ cycloalkyl. A² is optionally substituted as described previously.

In other embodiments, A² is selected from phenyl, pyridyl, thienyl, 1-oxidopyridinyl, and cyclohexyl. In many preferred embodiments, A² is phenyl. In either case, A² is substituted as described previously.

In many embodiments, R¹ is H or CH₃. In many embodiments, R¹ is H. In many embodiments, n is 0.

In many compounds of the invention, including pharmaceutically acceptable salts thereof, Z is selected from the group consisting of phenyl, tetrazolyl, oxadiazolyl, thiadiazolyl, 1,2,3-triazolyl, 1,2,4-triazolyl, oxazolyl, isoxazolyl, thiazolyl, isothiazolyl, pyrazolyl, imidazolyl, thienyl, furyl, pyridyl, pyrimidinyl, pyrazinyl, and dioxinyl, wherein Z is optionally substituted with 1-3 substituents independently selected from halogen, C₁-C₄ alkyl, —C₂-C₄ alkenyl, —C₂-C₄ alkynyl, —OC1-C₄alkyl, —OC₂-C₄ alkenyl, —OC₂-C₄ alkynyl, —C(═O)C₁-C₄alkyl, —CO₂C₁-C₄alkyl, —NR¹⁰R¹¹, —OH, —CN, and —NO₂, wherein the alkyl, alkenyl, and alkynyl groups of said substituents are optionally substituted with 1-5 halogens and optionally one substituent selected from —OH, —CO₂H, and —CO₂C₁-C₄alkyl.

In subsets of the compounds of Formula I and Ia, including pharmaceutically acceptable salts thereof, 2 groups R^{a} that are on adjacent carbon atoms of the phenyl or optional pyridinyl ring of Formula I and Ia do not have the option of joining to form a bridging moiety selected from —CH₂CH₂CH₂—, —CH₂CH₂CH₂CH₂—, and —CH═CH—CH═CH— to yield a cyclopentyl, cyclohexyl, or phenyl ring fused to the phenyl ring or optional pyridinyl ring of Formula I.

In subsets of the compounds of Formula I, including pharmaceutically acceptable salts thereof, the phenyl ring of Formula I does not have the option of having —N═ in place of one —CH═ of the phenyl ring.

In general, the compounds of the invention have at least one substituent other than H on at least two of the three rings (A¹, A², and the ring that is a phenyl ring which is optionally a pyridine ring and has A¹ connected to it), and more often have at least one substituent on each of the three rings. The ring A² often has 2 substituents other than H. The ring A¹ often has 2 substituents other than H, and in many preferred compounds has 3 substituents other than H.

A subset of compounds has the Formula II, shown below, and includes pharmaceutically acceptable salts thereof:

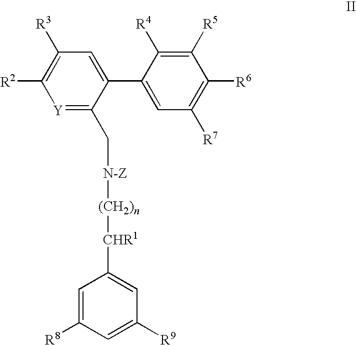

In the compound of Formula

Y is selected from —N═ and —CH═;

R¹ is selected from H and CH₃;

R² is selected from the group consisting of H, halogen, —NR¹⁰R¹¹, —OC₁-C₃ alkyl, C₁-C₃ alkyl, C₂-C₃ alkenyl, —CN, —NO₂, and pyridyl, wherein C₁-C₃ alkyl and C₂-C₃ alkenyl in all occurrences are optionally substituted with 1-3 halogens;

R³ is selected from the group consisting of H and —C₁-C₃ alkyl, which is optionally substituted with 1-3 F;

In the compound of formula II, R² and R³ do not have the option of joining to form a bridging group selected from —CH₂CH₂CH₂—, —CH₂CH₂CH₂CH₂—, and —CH═CH—CH═CH—;

R⁴ is selected from the group consisting of H, halogen, —C₁-C₃ alkyl, —OC₁-C₃ alkyl, —SC₁-C₂ alkyl and —CN, wherein —C₁-C₃ alkyl, —SC₁-C₃ alkyl, and —OC₁-C₃ alkyl are optionally substituted with 1-3 F;

R⁵ and R⁶ are each independently selected from the group consisting of H, halogen, —CH₃ and —OCH₃, wherein —CH₃ and —OCH₃ are optionally substituted with 1-3 F;

R⁷ is selected from the group consisting of H, —C₁-C₅alkyl, —OC₁-C₃ alkyl, —C₂-C₃ alkenyl, halogen, —CN, —CO₂H, —CO₂C₁-C₃ alkyl, —SC₁-C₃ alkyl, —C(═O)NR¹⁰R¹¹, —C(═O)H, —C(═O)C₁-C₃ alkyl, C₃-C₆ cycloalkyl, phenyl, and 5-(1,2,4-oxadiazolyl),

wherein —C₁-C₃ alkyl and —C₁-C₅ alkyl in all occurrences are optionally substituted with 1-6 substituent groups independently selected from 1-5 halogens and one —OH,

—C₂-C₃ alkenyl is optionally substituted with 1-3 halogens,

1,2,4-oxadiazolyl and C₃-C₆ cycloalkyl are optionally substituted with 1-2 substituent groups independently selected from halogen, C₁-C₃ alkyl, and CF₃, and

phenyl is optionally substituted with 1-3 substituents independently selected from halogen, —C₁-C₃ alkyl, —C₂-C₃ alkenyl, —OC₁-C₃ alkyl, —NR¹⁰R¹¹, —CO₂H, —CO₂C₁-C₃ alkyl, and —CN, wherein —C₁-C₃ alkyl and —C₂-C₃ alkenyl in all uses are optionally substituted with 1-3 halogens;

R⁸ and R⁹ are each independently selected from the group consisting of H, —C₁-C₃ alkyl, halogen, —S(O)ₓC₁-C₃ alkyl, —NR¹⁰R¹¹, —OC₁-C₃alkyl, C₂-C₃ alkenyl, —NO₂, —CN, —C(O)NR¹⁰R¹¹, —C(═O)H, —NHC(═O)C₁-C₃ alkyl, —NHS(O)₂C₁-C₃ alkyl, CO₂H, CO₂C₁₋₃alkyl, C₃-C₆ cycloalkyl, and pyridyl, wherein C₁-C₃ alkyl in all occurrences is optionally substituted with 1-3 halogens, C₂-C₃ alkenyl is optionally substituted with 1-3 halogens, and C₃-C₆ cycloalkyl and pyridyl are optionally substituted with 1-2 substituent groups independently selected from halogen and C₁-C₃ alkyl;

R¹⁰ and R¹¹ are each H or C₁-C₃ alkyl;

Z is selected from the group consisting of phenyl, tetrazolyl, 1,2,3-triazolyl, 1,2,4-triazolyl, thiadiazolyl, oxadiazolyl, oxazolyl, isoxazolyl, pyrazolyl, imidazolyl, thiazolyl, isothiazolyl, pyridyl, and pyrimidinyl, which are optionally substituted with 1-3 substituents independently selected from —CH₃ and —CF₃;

n is an integer selected from 0 and 1; and

x is an integer selected from 0, 1 and 2.

In subsets of the compound of Formula II, R² is selected from —OCF₃, —OCH₃, —NO₂, —CN, halogen, C₁-C₃alkyl, C₂-C₃alkenyl, —NH₂ and 3-pyridyl, wherein —C₁-C₃ alkyl and —C₂-C₃ alkenyl in all uses are optionally substituted with 1-3 F.

In subsets of the compound of Formula II, R³ is H or CH₃. In other subsets, R³ is H.

In subsets of the compound of Formula II, R⁴ is selected from the group consisting of H, halogen, C₁-C₃alkyl, C₂-C₃alkenyl, —OCH₃, —OCF₃, —OC₂H₅, —SCH₃, and —CN. In other subsets, R⁴ is selected from the group consisting of halogen, C₁-C₃alkyl, C₂-C₃alkenyl, —OCH₃, —OCF₃, —OC₂H₅, —SCH₃, and —CN.

In subsets of the compound of Formula II, R⁵ is H or F.

In subsets of the compound of Formula II, R⁶ is H, F, —CH₃, or —OCH₃. In other subsets, R⁶ is F.

In subsets of the compound of Formula II, R⁷ is selected from the group consisting of H, C₁-C₄alkyl, —C(═O)H, —C(═O)CH₃, —CH═CH₂, —CN, Cl, F, —CO₂H, —CO₂C₁-C₃alkyl, —OCH₃, —SCH₃, —C(═O)NR¹⁰R¹¹, 3-methyl-5-(1,2,4-oxadiazolyl), and phenyl, wherein C₁-C₄alkyl and C₁-C₃alkyl are optionally substituted with 1-6 substituents which are independently selected from 1-5 F and one —OH, and wherein phenyl is optionally substituted with 1-3 substituents independently selected from the group consisting of halogen, —C₁-C₃ alkyl, —C₂-C₃ alkenyl, —OC₁-C₃ alkyl, —NR¹⁰R¹¹, —CO₂H, —CO₂C₁-C₃ alkyl, and —CN, wherein —C₁-C₃ alkyl and —C₂-C₃ alkenyl in all uses are optionally substituted with 1-3 halogens. In other subsets, R⁷ is as described above, but is not H.

In subsets of the compound of Formula II, R⁸ and R⁹ are each independently selected from the group consisting of H, C₁-C₂alkyl, which is optionally substituted with 1-3 F; halogen; —CN; —NO₂; —S(O)ₓCH₃, which is optionally substituted with 1-3F; —OCH₃, which is optionally substituted with 1-3 F; —CH═CH₂; —C(═O)H; —C(═O)NR¹⁰R¹¹; —CO₂H; —NR¹⁰R¹¹; —CO₂C₁-C₃alkyl; —NHC(═O)CH₃; —NHS(O)₂CH₃; and 4-pyridyl.

In subsets of the compound of Formula II, R⁸ and R⁹ are CF₃.

In subsets of the compounds as described above, R¹⁰ and R¹¹ are each independently selected from H and CH₃.

In subsets of the compounds described above, R¹ is H, and n is 0.

In subsets of the compounds described above, or pharmaceutically acceptable salts thereof, Y is —CH═.

In subsets of the compounds described above, or pharmaceutically acceptable salts thereof, Y is —N═.

In subsets of the compounds described above, or pharmaceutically acceptable salts thereof, Z is selected from the group consisting of phenyl, tetrazolyl, 1,2,3-triazolyl, 1,2,4-triazolyl, thiadiazolyl, oxadiazolyl, oxazolyl, isoxazolyl, pyrazolyl, imidazolyl, thiazolyl, isothiazolyl, pyridyl, and pyrimidinyl, which are optionally substituted with 1-3 substituents independently selected from —CH₃ and —CF₃.

In subsets of the compounds described above, or pharmaceutically acceptable salts thereof, Z is selected from the group consisting of phenyl and a 5-6-membered heterocyclic ring having 1-3 heteroatoms independently selected from N, S, and O, said heterocyclic ring optionally also comprising 1-3 double bonds, said heterocyclic ring being connected by a carbon atom to the N to which Z is attached, wherein said phenyl and said 5-6-membered heterocyclic ring optionally comprises 1-3 substituents independently selected from halogen, C₁-C₄ alkyl, —C₂-C₄ alkenyl, —C₂-C₄ alkenyl, —OC₁-C₄alkyl, —OC₂-C₄ alkenyl, —OC₂-C₄ alkynyl, —C(═O)C₁-C₄alkyl, —CO₂C₁-C₄alkyl, —NR¹⁰R¹¹, —OH, —CN, and —NO₂, wherein the alkyl, alkenyl, and alkynyl groups of said substituents are optionally substituted with 1-5 halogens and optionally one substituent selected from —OH, —CO₂H, and —CO₂C₁-C₄alkyl.

In subsets of the compounds described above, or pharmaceutically acceptable salts thereof, Z is selected from the group consisting of phenyl and a 5-6-membered heteroaromatic ring having 1-4 heteroatoms independently selected from N, S, and O, wherein said heteroaromatic ring is connected by a carbon atom to the N to which Z is attached, wherein said phenyl and said 5-6-membered heteroaromatic ring optionally comprise 1-3 substituents independently selected from halogen, C₁-C₃ alkyl and —OC₁-C₃alkyl, wherein C₁-C₃ alkyl and —OC₁-C₃alkyl are optionally substituted with 1-3 halogens.

In subsets of the compounds described above, or pharmaceutically acceptable salts thereof, Z is selected from the group consisting of phenyl and a 5-6-membered heteroaromatic ring having 1-3 heteroatoms independently selected from N, S, and O, wherein said heteroaromatic ring is connected by a carbon atom to the N to which Z is attached, wherein said phenyl and said 5-6-membered heteroaromatic ring optionally comprise 1-3 substituents independently selected from halogen, C₁-C₃ alkyl and —OC₁-C₃alkyl, wherein C₁-C₃ alkyl and —OC₁-C₃alkyl are optionally substituted with 1-3 halogens.

In subsets of the compounds described above, or pharmaceutically acceptable salts thereof, Z is a 5-6-membered heteroaromatic ring having 1-3 heteroatoms independently selected from N, S, and O, wherein said heteroaromatic ring is connected by a carbon atom to the N to which Z is attached, wherein said 5-6-membered heteroaromatic ring optionally comprises 1-3 substituents independently selected from halogen, C₁-C₃ alkyl and —OC₁-C₃alkyl, wherein C₁-C₃ alkyl and —OC₁-C₃alkyl are optionally substituted with 1-3 halogens.

In subsets of the compounds described above, or pharmaceutically acceptable salts thereof, Z is selected from the group consisting of tetrazolyl, isoxazolyl, triazolyl, pyrazolyl, oxadiazolyl, and thiadiazolyl, which are optionally substituted with 1-3 substituents independently selected from halogen, —CH₃, —OCH₃, —CF₃, and —OCF₃.

In subsets of the compounds described above, or pharmaceutically acceptable salts thereof, Z is selected from the group consisting of phenyl, isoxazolyl, and triazolyl, wherein Z is optionally substituted with 1-3 substituents independently selected from halogen, —CH₃, —OCH₃, —CF₃, and —OCF₃.

In subsets of the compounds described above, or pharmaceutically acceptable salts thereof, Z is selected from the group consisting of isoxazolyl, and triazolyl, wherein Z is optionally substituted with 1-3 substituents independently selected from halogen, —CH₃, —OCH₃, —CF₃, and —OCF₃.

In subsets of the compounds described above, or pharmaceutically acceptable salts thereof, Z is selected from the group consisting of phenyl, isoxazol-3-yl, isoxazol-5-yl, and 1,2,3-triazol-4-yl, which are optionally substituted with 1-2 substituents independently selected from halogen, —CH₃, —OCH₃, —CF₃, and —OCF₃.

In subsets of the compounds described above, or pharmaceutically acceptable salts thereof, Z is selected from the group consisting of isoxazol-3-yl, isoxazol-5-yl, and 1,2,3-triazol-4-yl, which are optionally substituted with 1-2 substituents independently selected from halogen, —CH₃, —OCH₃, —CF₃, and —OCF₃.

In subsets of the compounds described above, or pharmaceutically acceptable salts thereof, Z is phenyl, which is optionally substituted with 1-3 substituents independently selected from halogen, —CH₃, —OCH₃, —CF₃, and —OCF₃.

In subsets of the compounds described above, or pharmaceutically acceptable salts thereof, Z is tetrazolyl, which is optionally substituted with one substituent selected from halogen, —CH₃, —OCH₃, —CF₃, and —OCF₃.

In subsets of compounds of Formula Ia, Ib, Ic, or II, Y is —N═ or —CH═; except that when Z is tetrazolyl, Y is —N═.

In subsets of compounds of Formula Ia, Ib, Ic, or II, Y is —N═ or —CH═, except that when Z is a heteroaromatic group, then Y is —N═.

### DEFINITIONS

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkylene” groups are alkyl groups that are difunctional rather than monofunctional. For example, methyl is an alkyl group and methylene (—CH₂—) is the corresponding alkylene group.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means a saturated carbocyclic ring having from 3 to 8 carbon atoms, unless otherwise stated. The term also includes a cycloalkyl ring fused to an aryl group. Examples of cycloalkyl include cyclopropyl, cyclopentyl, cyclohexyl, cycloheptyl, and the like. “Cycloalkyl” may also be defined to have one or more double bonds, such as cyclohexenyl or cyclohexadienyl, but cannot have the number of double bonds that would make the cycloalkyl group aromatic.

“Aryl” (and “arylene”) when used to describe a substituent or group in a structure means a monocyclic or bicyclic compound in which the rings are aromatic and which contains only carbon ring atoms. The term “aryl” can also refer to an aryl group that is fused to a cycloalkyl or heterocycle. Preferred “aryls” are phenyl and naphthyl. Phenyl is generally the most preferred aryl group.

“Heterocyclyl,” “heterocycle,” and “heterocyclic” means a fully or partially saturated or aromatic 5-6 membered ring containing 1-4 heteroatoms in the ring independently selected from N, S and O, unless otherwise stated. The heterocyclic ring may also be defined to include an optional carbonyl group or —N(O)-group as part of the ring structure. An example of the latter is pyridine N-oxide.

“Benzoheterocycle” represents a phenyl ring fused to a 5-6-membered heterocyclic ring having 1-2 heteroatoms, each of which is O, N, or S, where the heterocyclic ring may be saturated or unsaturated (i.e. the heterocyclic ring may have 1-2 double bonds in addition to the double bond of the phenyl ring). Examples include indole, 2,3-dihydroindole, benzofuran, 2,3-dihydrobenzofuran, quinoline, and isoquinoline. When the fused heterocycle is aromatic, the benzoheterocycle may also be referred to as benzoheteroaromatic or benzheteroaryl.

“Halogen” includes fluorine, chlorine, bromine and iodine. Halogen substitutents are most often fluorine or chlorine.

“Me” represents methyl.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The substituent “tetrazole” means a 2H-tetrazol-5-yl substituent group and tautomers thereof.

### Optical Isomers—Diastereomers—Geometric Isomers—Tautomers

Compounds of Formula I may contain one or more asymmetric centers and can thus occur as racemates, racemic mixtures, single enantiomers, diastereomeric mixtures and individual diastereomers. The present invention is meant to include all such isomeric forms of the compounds of Formula I and all mixtures of the compounds. When structures are shown with a stereochemical representation, other stereochemical structures are also included individually and collectively, such as enantiomers, diastereoisomers (where diastereomers are possible), and mixtures of the enantiomers and/or diastereomers, including racemic mixtures.

Some of the compounds described herein may contain olefinic double bonds, and unless specified otherwise, are meant to include both E and Z geometric isomers.

Some of the compounds described herein may exist as tautomers. An example is a ketone and its enol form, known as keto-enol tautomers. The individual tautomers as well as mixtures thereof are encompassed with compounds of Formula I.

Compounds of Formula I having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

Some of the biphenyl and biaryl compounds herein may comprise mixtures of atropisomers (rotamers) in the NMR spectra. The individual atropisomers as well as mixtures thereof are encompassed with the compounds of this invention.

### Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

### Metabolites—Prodrugs

Therapeutically active metabolites, where the metabolites themselves fall within the scope of the claimed invention, are also compounds of the current invention. Prodrugs, which are compounds that that are converted to the claimed compounds as they are being administered to a patient or after they have been administered to a patient, are also compounds of this invention.

### Utilities

Compounds of the current invention are potent inhibitors of CETP. They are therefore useful in treating diseases and conditions that are treated by inhibitors of CETP.

One aspect of the present invention provides a method for treating or reducing the risk of developing a disease or condition that may be treated or prevented by inhibition of CETP by administering a therapeutically effective amount of a compound of this invention to a patient in need of treatment. A patient is a human or mammal, and is most often a human. A “therapeutically effective amount” is the amount of compound that is effective in obtaining a desired clinical outcome in the treatment of a specific disease.

Diseases or conditions that may be treated with compounds of this invention, or which the patient may have a reduced risk of developing as a result of being treated with the compounds of this invention, include: atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension, vascular complications of diabetes, obesity, endotoxemia, and metabolic syndrome.

The compounds of this invention are expected to be particularly effective in raising HDL-C and/or increasing the ratio of HDL-C to LDL-C. These changes in HDL-C and LDL-C may be beneficial in treating atherosclerosis, reducing or reversing the development of atherosclerosis, reducing the risk of developing atherosclerosis, or preventing atherosclerosis.

### Administration and Dose Ranges

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating the diseases for which compounds of Formula I are indicated, generally satisfactory results are obtained when the compounds of the present invention are administered at a daily dosage of from about 0.01 milligram to about 100 milligram per kilogram of animal or human body weight, preferably given as a single daily dose or in divided doses two to six times a day, or in sustained release form. In the case of a 70 kg adult human, the total daily dose will generally be from about 0.5 milligram to about 500 milligrams. For a particularly potent compound, the dosage for an adult human may be as low as 0.1 mg. The dosage regimen may be adjusted within this range or even outside of this range to provide the optimal therapeutic response.

Oral administration will usually be carried out using tablets. Examples of doses in tablets are 0.5 mg, 1 mg, 2 mg, 5 mg, 10 mg, 25 mg, 50 mg, 100 mg, 250 mg, and 500 mg. Other oral forms can also have the same dosages (e.g. capsules).

### Pharmaceutical Compositions

Another aspect of the present invention provides pharmaceutical compositions which comprise a compound of Formula I and a pharmaceutically acceptable carrier. The pharmaceutical compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered. Pharmaceutical compositions may also consist essentially of a compound of Formula I and a pharmaceutically acceptable carrier without other therapeutic ingredients.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

### Combination Therapy

Compounds of the invention (e.g. Formula I and Ia-Ij) may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different schedules.

When oral formulations are used, the drugs may be combined into a single combination tablet or other oral dosage form, or the drugs may be packaged together as separate tablets or other oral dosage forms. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of this invention (e.g. Formula I), and either administered separately or in the same pharmaceutical composition, include, but are not limited to, other compounds which improve a patient's lipid profile, such as (i) HMG-CoA reductase inhibitors, (which are generally statins, including lovastatin, simvastatin, rosuvastatin, pravastatin, fluvastatin, atorvastatin, rivastatin, itavastatin, pitavastatin, and other statins), (ii) bile acid sequestrants (cholestyramine, colestipol, dialkylaminoalkyl derivatives of a cross-linked dextran, Colestid®, LoCholest®, (iii) niacin and related compounds, such as nicotinyl alcohol, nicotinamide, and nicotinic acid or a salt thereof, (iv) PPARα agonists, such as gemfibrozil and fenofibric acid derivatives (fibrates), including clofibrate, fenofibrate, bezafibrate, ciprofibrate, and etofibrate, (v) cholesterol absorption inhibitors, such as stanol esters, beta-sitosterol, sterol glycosides such as tiqueside; and azetidinones, such as ezetimibe, (vi) acyl CoA:cholesterol acyltransferase (ACAT) inhibitors, such as avasimibe and melinamide, and including selective ACAT-1 and ACAT-2 inhibitors and dual inhibitors, (vii) phenolic anti-oxidants, such as probucol, (viii) microsomal triglyceride transfer protein (MTP)/ApoB secretion inhibitors, (ix) anti-oxidant vitamins, such as vitamins C and E and beta carotene, (x) thyromimetics, (xi) LDL (low density lipoprotein) receptor inducers, (xii) platelet aggregation inhibitors, for example glycoprotein IIb/IIIa fibrinogen receptor antagonists and aspirin, (xiii) vitamin B12 (also known as cyanocobalamin), (xiv) folic acid or a pharmaceutically acceptable salt or ester thereof, such as the sodium salt and the methylglucamine salt, (xv) FXR and LXR ligands, including both inhibitors and agonists, (xvi) agents that enhance ABCA1 gene expression, and (xvii) ileal bile acid transporters.

Preferred classes of therapeutic compounds that can be used with the compounds of this invention for use in improving a patient's lipid profile (i.e. raising HDL-C and lowering LDL-C) include one or both of statins and cholesterol absorption inhibitors. Particularly preferred are combinations of compounds of this invention with simvastatin, ezetimibe, or both simvastatin and ezetimibe. Also preferred are combinations of compounds of this invention with statins other than simvastatin, such as lovastatin, rosuvastatin, pravastatin, fluvastatin, atorvastatin, rivastatin, itavastatin, and ZD-4522.

Finally compounds of this invention can be used with compounds that are useful for treating other diseases, such as diabetes, hypertension and obesity, as well as other anti-atherosclerostic compounds. Such combinations may be used to treat one or more of such diseases as diabetes, obesity, atherosclerosis, and dyslipidemia, or more than one of the diseases associated with metabolic syndrome. The combinations may exhibit synergistic activity in treating these disease, allowing for the possibility of administering reduced doses of active ingredients, such as doses that otherwise might be sub-therapeutic.

Examples of other active ingredients that may be administered in combination with a compound of this invention include, but are not limited to, compounds that are primarily anti-diabetic compounds, including:

(a) PPAR gamma agonists and partial agonists, including glitazones and non-glitazones (e.g. pioglitazone, englitazone, MCC-555, rosiglitazone, balaglitazone, netoglitazone, T-131, LY-300512, and LY-818;

(b) biguanides such as metformin and phenformin;

(c) protein tyrosine phosphatase-1B (PTP-1B) inhibitors;

(d) dipeptidyl peptidase IV (DP-IV) inhibitors, including vildagliptin, sitagliptin, and saxagliptin;

(e) insulin or insulin mimetics, such as for example insulin lispro, insulin glargine, insulin zinc suspension, and inhaled insulin formulations;

(f) sulfonylureas, such as tolbutamide, glipizide, glimepiride, acetohexamide, chlorpropamide, glibenclamide, and related materials;

(g) α-glucosidase inhibitors (such as acarbose, adiposine; camiglibose; emiglitate; miglitol; voglibose; pradimicin-Q; and salbostatin);

(h) PPARα/γ dual agonists, such as muraglitazar, tesaglitazar, farglitazar, and naveglitazar;

(i) PPARδ agonists such as GW501516 and those disclosed in WO97/28149;

(j) glucagon receptor antagonists;

(k) GLP-1; GLP-1 derivatives; GLP-1 analogs, such as exendins, such as for example exenatide (Byetta); and non-peptidyl GLP-1 receptor agonists;

(l) GIP-1; and

(m) Non-sulfonylurea insulin secretagogues, such as the meglitinides (e.g. nateglinide and rapeglinide).

These other active ingredients that may be used in combination with the current invention also include antiobesity compounds, including 5-HT(serotonin) inhibitors, neuropeptide Y5 (NPY5) inhibitors, melanocortin 4 receptor (Mc4r) agonists, cannabinoid receptor 1 (CB-1) antagonists/inverse agonists, and β₃ adrenergic receptor agonists. These are listed in more detail later in this section.

These other active ingredients also include active ingredients that are used to treat inflammatory conditions, such as aspirin, non-steroidal anti-inflammatory drugs, glucocorticoids, azulfidine, and selective cyclooxygenase-2 (COX-2) inhibitors, including etoricoxib, celecoxib, rofecoxib, and Bextra.

Antihypertensive compounds may also be used advantageously in combination therapy with the compounds of this invention. Examples of antihypertensive compounds that may be used with the compounds of this invention include (1) angiotensin II antagonists, such as losartan; (2) angiotensin converting enzyme inhibitors (ACE inhibitors), such as enalapril and captopril; (3) calcium channel blockers such as nifedipine and diltiazam; and (4) endothelian antagonists.

Anti-obesity compounds may be administered in combination with the compounds of this invention, including: (1) growth hormone secretagogues and growth hormone secretagogue receptor agonists/antagonists, such as NN703, hexarelin, and MK-0677; (2) protein tyrosine phosphatase-1B (PTP-1B) inhibitors; (3) cannabinoid receptor ligands, such as cannabinoid CB₁ receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer); (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A; (6) pancreatic lipase inhibitors, such as orlistat (Xenical®), Triton WR1339, RHC80267, lipstatin, tetrahydrolipstatin, teasaponin, and diethylumbelliferyl phosphate; (7) neuropeptide Y1 antagonists, such as BIBP3226, J-115814, BIBO 3304, LY-357897, CP-671906, and GI-264879A; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104; (9) melanin-concentrating hormone (MCH) receptor antagonists; (10) melanin-concentrating hormone 1 receptor (MCH1R) antagonists, such as T-226296 (Takeda); (11) melanin-concentrating hormone 2 receptor (MCH2R) agonist antagonists; (12) orexin-1 receptor antagonists, such as SB-334867-A; (13) melanocortin agonists, such as Melanotan II; (14) other Mc4r (melanocortin 4 receptor) agonists, such as CHIR86036 (Chiron), ME-10142, and ME-10145 (Melacure), CHIR86036 (Chiron); PT-141, and PT-14 (Palatin); (15) 5HT-2 agonists; (16) 5HT2C (serotonin receptor 2C) agonists, such as BVT933, DPCA37215, WAY161503, and R-1065; (17) galanin antagonists; (18) CCK agonists; (19) CCK-A (cholecystokinin-A) agonists, such as AR-R 15849, GI 181771, JMV-180, A-71378, A-71623 and SR146131; (20) GLP-1 agonists; (21) corticotropin-releasing hormone agonists; (22) histamine receptor-3 (H3) modulators; (23) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, and GT2394 (Gliatech); (24) (3-hydroxy steroid dehydrogenase-1 inhibitors (11β-HSD-1 inhibitors), such as BVT 3498 and, BVT 2733, (25) PDE (phosphodiesterase) inhibitors, such as theophylline, pentoxifylline, zaprinast, sildenafil, aminone, milrinone, cilostamide, rolipram, and cilomilast; (26) phosphodiesterase-3B (PDE3B) inhibitors; (27) NE (norepinephrine) transport inhibitors, such as GW 320659, despiramine, talsupram, and nomifensine; (28) ghrelin receptor antagonists; (29) leptin, including recombinant human leptin (PEG-OB, Hoffman La Roche) and recombinant methionyl human leptin (Amgen); (30) leptin derivatives; (31) BRS3 (bombesin receptor subtype 3) agonists such as [D-Phe6,beta-Ala11,Phe13,Nle14]Bn(6-14) and [D-Phe6,Phe13]Bn(6-13)propylamide; (32) CNTF (Ciliary neurotrophic factors), such as GI-181771 (Glaxo-SmithKline), SR146131 (Sanofi Synthelabo), butabindide, PD170,292, and PD 149164 (Pfizer); (33) CNTF derivatives, such as axokine (Regeneron); (34) monoamine reuptake inhibitors, such as sibutramine; (35) UCP-1 (uncoupling protein-1, 2, or 3) activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; (36) thyroid hormone β agonists, such as KB-2611 (KaroBioBMS); (37) FAS (fatty acid synthase) inhibitors, such as Cerulenin and C75; (38) DGAT1 (diacylglycerol acyltransferase 1) inhibitors; (39) DGAT2 (diacylglycerol acyltransferase 2) inhibitors; (40) ACC2 (acetyl-CoA carboxylase-2) inhibitors; (41) glucocorticoid antagonists; (42) acyl-estrogens, such as oleoyl-estrone; (43) dicarboxylate transporter inhibitors; (44) peptide YY, PYY 3-36, peptide YY analogs, derivatives, and fragments such as BIM-43073D, BIM-43004C, (45) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (46) Neuropeptide Y4 (NPY4) agonists such as pancreatic peptide (PP); (47) Neuropeptide Y1 (NPY1) antagonists such as B1BP3226, J-115814, BIBO 3304, LY-357897, CP-671906, and GI-264879A; (48) Opioid antagonists, such as nalmefene (Revex®), 3-methoxynaltrexone, naloxone, and naltrexone; (49) glucose transporter inhibitors; (50) phosphate transporter inhibitors; (51) 5-HT (serotonin) inhibitors; (52) beta-blockers; (53) Neurokinin-1 receptor antagonists (NK-1 antagonists); (54) clobenzorex; (55) cloforex; (56) clominorex; (57) clortermine; (58) cyclexedrine; (59) dextroamphetamine; (60) diphemethoxidine, (61) N-ethylamphetamine; (62) fenbutrazate; (63) fenisorex; (64) fenproporex; (65) fludorex; (66) fluminorex; (67) furfurylmethylamphetamine; (68) levamfetamine; (69) levophacetoperane; (70) mefenorex; (71) metamfepramone; (72) methamphetamine; (73) norpseudoephedrine; (74) pentorex; (75) phendimetrazine; (76) phenmetrazine; (77) picilorex; (78) phytopharm 57; (79) zonisamide, (80) a minorex; (81) amphechloral; (82) amphetamine; (83) benzphetamine; and (84) chlorphentermine.

The combination therapies described above which use the compounds of this invention may also be useful in the treatment of the metabolic syndrome. According to one widely used definition, a patient having metabolic syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined clinically in the recently released Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with metabolic syndrome have an increased risk of developing the macrovascular and microvascular complications that are listed above, including atherosclerosis and coronary heart disease. The combinations described above may ameliorate more than one symptom of metabolic syndrome concurrently (e.g. two symptoms, three symptoms, four symptoms, or all five of the symptoms).

### CETP ASSAY

An in vitro continuous assay for determining IC₅₀'s to identify compounds that are CETP inhibitors was performed based on a modification of the method described by Epps et al. employing BODIPY®-CE as the cholesteryl ester lipid donor. See Epps et al. (1995) Method for measuring the activities of cholesteryl ester transfer protein (lipid transfer protein), Chem. Phys. Lipids. 77, 51-63.

Particles used in the assay were created from the following sources: Synthetic donor HDL particles containing DOPC (Dioleoyl Phosphatidyl Choline), BODIPY®-CE (Molecular Probes C-3927), triolein (a triglyceride), and apoHDL were essentially created by probe sonication as described by Epps et al, but with the addition of a non-diffusable quencher molecule, dabcyl dicetylamide, in order to reduce background fluorescence. Dabcyl dicetylamide was made by heating dabcyl n-succinimide with dicetylamine in DMF at 95° C. overnight in the presence of diisopropylamine catalyst. Native lipoproteins from human blood were used as acceptor particles. Particles having a density less than 1.063 g/ml were collected by ultracentrifugation. These particles include VLDL, IDL, and LDL. Particle concentrations were expressed in terms of protein concentration as determined by BCA assay (Pierce, USA). Particles were stored at 4° C. until use.

Assays were performed in Dynex Microfluor 2 U-bottom black 96-well plates (Cat #7205). An assay cocktail containing CETP, 1× CETP buffer (50 mM Tris, pH 7.4, 100 mM NaCl, 1 mM EDTA), and half the final concentration of acceptor particles was prepared, and 100 μL of the assay cocktail was added to each well of the plate. Test compounds in DMSO were added in a volume of 3 μL. The plate was mixed on a plate shaker and then incubated at 25° C. for 1 hour. A second assay cocktail containing donor particles, the remaining acceptor particles and 1× CETP buffer was prepared. 47 μL of the second assay cocktail was added to the reaction wells to start the assay. Assays were performed at 25° C. in a final volume of 150 μL. Final concentrations of materials were: 5 ng/μL donor particles, 30 ng/μL acceptor particles (each expressed by protein content), 1× CETP buffer, 0.8 nM recombinant human CETP (expressed in CHO cells and partially purified), and up to 2% DMSO when testing compounds. The assay was followed in a fluorescence plate reader (Molecular Devices Spectramax GeminiXS) set for a 45 minute kinetic run at 25° C. which read the samples every 45 sec at Ex=480 nm, Em=511 nm, with a cutoff filter at 495 nm, photomultiplier tube setting of medium, calibration on, and 6 reads/well.

Data was evaluated by obtaining an initial rate, expressed in relative fluorescence units per second, for the pseudolinear portion of the curve, often 0-500 or 1000 sec. Comparison of the rates of samples with inhibitors to an uninhibited (DMSO only) positive control yielded a percent inhibition. A plot of percent inhibition vs. log of inhibitor concentration, fit to a Sigmoidal 4 parameter equation was used to calculate IC₅₀.

### EXAMPLES

The following schemes and examples are provided so that the invention will be more fully appreciated and understood. Starting materials are made using known procedures or as shown below.

The examples should not be construed as limiting the invention in any way. The scope of the invention is defined only by the appended claims.

Compounds of this invention have an IC₅₀ value as measured using the assay described above of less than or equal to 50 μM, preferably less than 10 μM, and more preferably less than 1 μM. Compounds described in the examples have an IC₅₀ value in the range of about 13 μM to about 300 μM.

Several methods for preparing the compounds in this invention are illustrated in the following Schemes and Examples. Starting materials are made from known procedures or as illustrated.

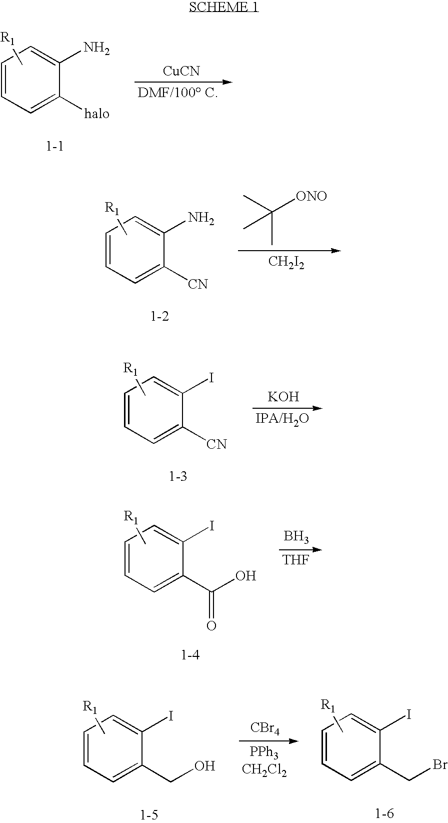

Intermediates 1-2, 1-3 and 1-4 utilized in the present invention can be purchased or prepared as shown in Scheme 1. An appropriately substituted 2-haloaniline 1-1 where the halogen is preferably iodo or bromo is treated with CuCN in DMF at elevated temperature to afford the corresponding 2-cyanoaniline 1-2. Alternatively, the nitrile can be prepared by treatment of 1-1 with KCN and CuI in the presence of a palladium (II) salt or in the presence of certain copper or nickel complexes (See: Smith, M. B. and March, J. “March's Advanced Organic Chemistry”, 5^{th} Ed., John Wiley and Sons, New York, pp. 867 (2001) and references therein). Iodides 1-3 are prepared by treatment of 1-2 with isoamylnitrite, n-pentylnitrite, t-butyl nitrite or the like in diiodomethane (see for example: Smith et al., J. Org. Chem. 55, 2543, (1990) and references cited therein). Alternatively, the iodide can be prepared first by diazonium formation using isoamylnitrite, n-pentylnitrite, t-butyl nitrite, sodium nitrite, nitrous acid or the like followed by heating in the presence of an iodide salt such as copper iodide, sodium iodide, potassium iodide, tetrabutylammonium iodide or the like. Hydrolysis of iodo-nitrile 1-3 is carried out using potassium hydroxide in isopropanol and water to afford the iodoacid 1-4. Further reduction with borane, lithium aluminum hydride, lithium borohydride or the like in ether, tetrahydrofuran, dimethoxyethane or the like affords the 2-iodo alcohols 1-5. Intermediates 1-5 can be transformed into benzyl bromides 1-6 using reagents such as triphenylphosphine and carbon tetrabromide in solvents such as dichloromethane or the like (see Smith, M. B. and March, J. “March's Advanced Organic Chemistry”, 5^{th} Ed., John Wiley and Sons, New York, pp. 518-199 (2001) and references therein).

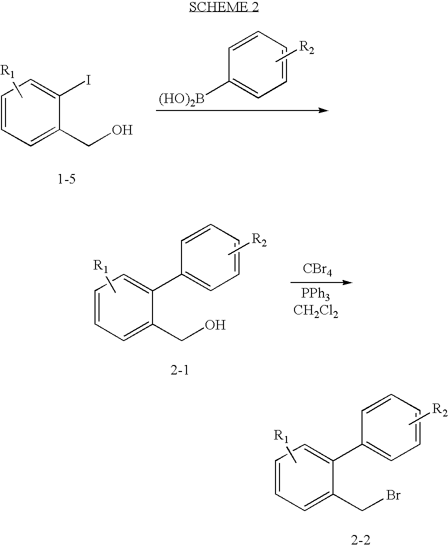

Intermediates 2-2 utilized in the present invention can be prepared as shown in Scheme 2. Benzyl alcohols 1-5 can be purchased or prepared according to the procedure outlined in Scheme 1. Intermediates 2-1 can be prepared via Suzuki reaction wherein 1-5 is coupled with an appropriately substituted aryl boronic acid or aryl boronate ester in the presence of a palladium catalyst. The coupling reaction may be carried out using Pd(II) acetate and potassium carbonate in aqueous acetone at reflux. Alternatively the reaction may employ tetrakis(triphenylphosphine)palladium in an ethanol/toluene mix in the presence of sodium carbonate. Alternatively, as practiced by those skilled in the art the reaction can employ a number of Palladium (0) compounds and Palladium (II) salts in a number of solvents and in the presence of a variety of ligands, bases, and promoters, generally but not exclusively, with heating and/or microwave irradiation. Some appropriate reaction conditions can be found described in Miyaua et al., Chem. Rev. 95, 2457 (1995) and references cited within and as described in Smith, M. B. and March, J. “March's Advanced Organic Chemistry”, 5^{th} Ed., John Wiley and Sons, New York, pp. 868-869 (2001) and references cited therein. Compounds 2-2 are prepared from intermediates 2-1 as described in Scheme 1.

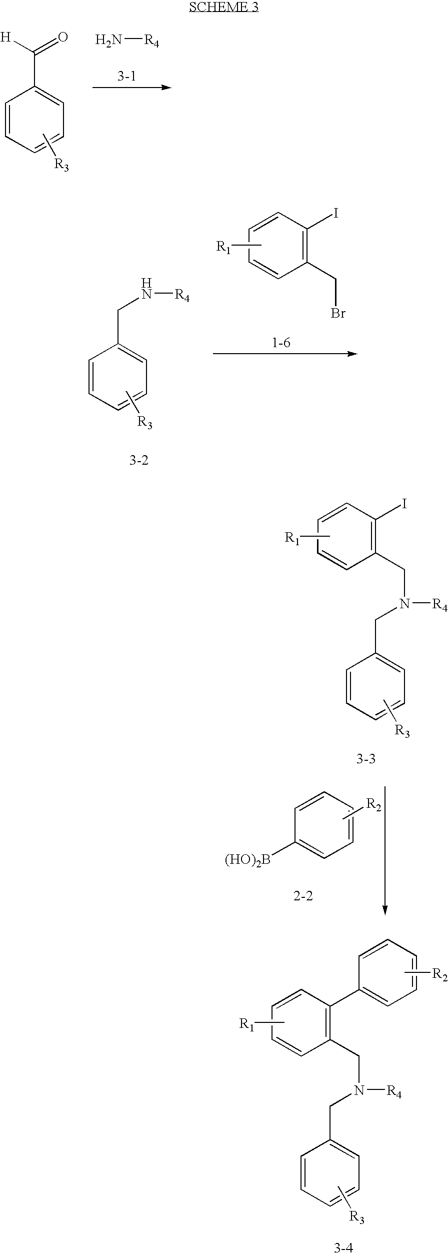

Compounds of the present invention can be prepared as shown in Scheme 3. Benzylamines 3-2 can be prepared by treatment of an amine with an appropriately substituted benzaldehyde in the presence of a reducing agent such as sodium borohydride, sodium cyanoborohydride, sodium triacetoxyborohydride or the like in methanol, ethanol, dichloroethane, tetrahydrofuran or the like or according to methods described in Smith, M. B. and March, J. “March's Advanced Organic Chemistry”, 5^{th} Ed., John Wiley and Sons, New York, pp. 1187-1189 (2001) and references cited therein. Alkylation of 3-1 can be carried out by treatment with an appropriately substituted benzyl halide, mesylate or tosylate or the like in dichloromethane, dichloroethane, tetrahydrofuran, dimethoxyethane or the like in the presence of a base such as triethylamine, diisopropylethylamine, N-methylmorpholine, lithium diisopropylamide or lithium-, sodium-, or potassium bis(trimethylsilyl)amide or the like to afford dibenzylamine 3-3. Biarylamines 3-4 can be prepared from intermediates 3-3 via Suzuki reaction as described previously in Scheme 2. Amines 3-1 were obtained from commercial sources or prepared from known procedures. For example, 5-amino-1H-tetrazole and 2-methyl-5-amino tetrazole were prepared according to the procedures described in J. Am. Chem. Soc. 1954, 76, 923-926 and J. Am. Chem. Soc. 1956, 78, 411-415, respectively.

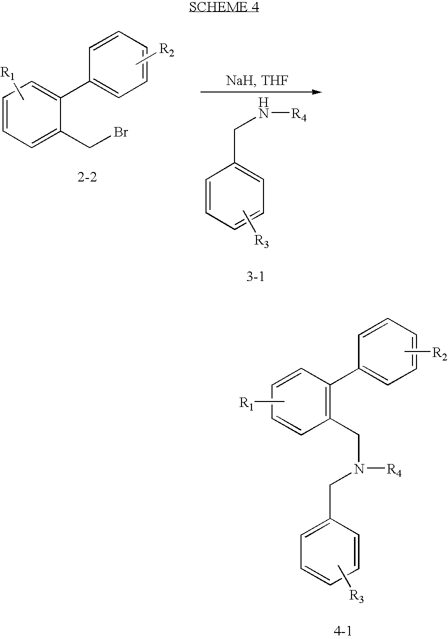

Compounds of the present invention can be prepared as shown in Scheme 4. 2-halobenzylbromides 2-2 wherein the halo is preferably iodo or bromo can be purchased or prepared as described in Schemes 1 and 2. Treatment of 2-2 with an appropriately substituted benzylamine such as 3-1 in the presence of a base such as sodium hydride or potassium tert-butoxide or the like in tetrahydrofuran, DMF or the like affords biaryl benzylamines 4-1.

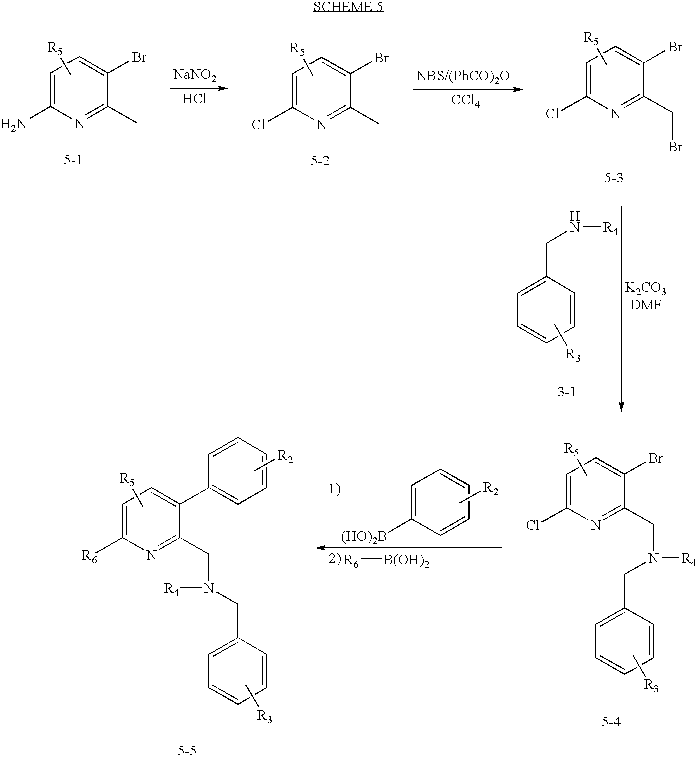

Heterocyclic biarylamines 5-5 can be prepared as shown in Scheme 5. An appropriately substituted amino pyridine 5-1 can be converted to the corresponding chloro pyridine 5-2 by diazonium formation using isoamylnitrite, n-pentylnitrite, t-butyl nitrite, sodium nitrite, nitrous acid or the like followed by treatment with concentrated HCl. Subsequent bromination of 5-2 is carried out using N-bromosuccinimide and benzoyl peroxide in solvents such as carbon tetrachloride and the like to afford the benzyl bromide 5-3. Other methods for benzylic halogenation can be found in Smith, M. B. and March, J. “March's Advanced Organic Chemistry”, 5^{th} Ed., John Wiley and Sons, New York, pp. 911 (2001) and references cited therein. Conversion to the benzylic amines 5-4 and 5-5 can be carried out via alkylation with benzylamine 3-1 followed by sequential Suzuki reaction, respectively, as described previously in Schemes 2 and 3.

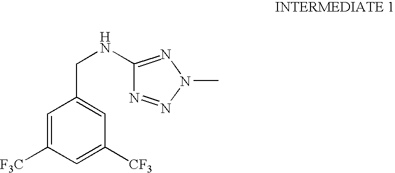

### N-[3,5-bis(trifluoromethyl)benzyl]-2-methyl-2H-tetrazol-5-amine

A stirred solution of 3,5-bis(trifluoromethyl)benzaldehyde (877 μL, 5.29 mmol) and 2-methyl-2H-tetrazol-5-amine (628 mg, 6.35 mmol) in toluene (15 mL) was heated at reflux for 2.5 h. The reaction was concentrated in vacuo and the residue was redissolved in EtOH (15 mL). Sodium borohydride (400 mg, 10.58 mmol) was added and the mixture was stirred at room temperature for 14 h. The reaction was quenched with sat. NH₄Cl and was partitioned between H₂O (25 mL) and EtOAC (25 mL). The aqueous layer was re-extracted with EtOAc (3×25 mL) and the combined extracts were washed with brine (50 mL), dried (Na₂SO₄), filtered and concentrated in vacuo. The residue was recrystallized from IPA:H₂O (3:7) and cooled at 4° C. for 14 h. A precipitate formed and was collected by filtration and dried in a vacuum oven to afford N-[3,5-bis(trifluoromethyl)benzyl]-2-methyl-2H-tetrazol-5-amine as a white solid. LCMS=326.1 (M+1)⁺. ¹H NMR (CDCl₃, 500 MHz): δ 7.86 (s, 2H), 7.82 (s, 1 II), 4.69 (s, 2H), 4.19 (s, 3H).

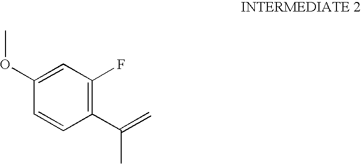

### 2-fluoro-1-isopropenyl-4-methoxybenzene

### Step A: 2-(2-fluoro-4-methoxyphenyl)propan-2-ol

To a solution of 2-fluoro-4-methoxyacetophenone (4.45 g, 26.5 mmol) in THF (50 mL) at 0° C., a solution of 2.4 M MeMgBr (11.6 mL, 27.8 mmol) was added. The mixture was stirred at 0° C. and then room temperature for 4 h. The reaction was quenched with saturated ammonium chloride solution. The organic layer was extracted with ethyl acetate (3×50 mL). The combined ethyl acetate layers were washed with brine and dried over sodium sulfate. The title compound was obtained as an oil after flash column using EtOAc:hexane=2:8 as the elute.

### Step B: 2-fluoro-1-isopropenyl-4-methoxybenzene

To a solution of 2-(2-fluoro-4-methoxyphenyl)propan-2-ol from Step A (3.89 g, 21.14 mmol) in methylene chloride (50 mL) at 0° C., MsCl (1.95 mL, 25.4 mmol) and triethylamine (6.52 mL, 46.5 mmol) were added. The solution was stirred at 0° C. and then room temperature for 2 h. The solution was diluted with methylene chloride (100 mL), washed with water, and dried over sodium sulfate. The title compound was obtained as an oil after flash column using EtOAc:hexane=1:9 as the elute. ¹H NMR (CDCl₃, 500 MHz) δ 7.25 (t, J=9.0 Hz, 1H), 6.68 (dd, J=8.5, 2.5 Hz, 1H), 6.63 (dd, J=13, 2.5 Hz, 1H), 5.20 (d, J=17.0 Hz, 2H), 3.82 (s, 3H), 2.18 (s, 3H).

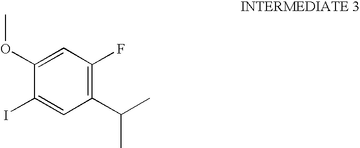

### 1-fluoro-4-iodo-2-isopropyl-5-methoxybenzene

A solution of the 2-fluoro-1-isopropenyl-4-methoxybenzene (Intermediate 2, 1.96 g, 11.81 mmol) in MeOH (30 mL) was charged with hydrogen at 1 atm and a catalytic amount of Pd/C. The mixture was stirred at room temperature for 1 h. The mixture was filtered through Celite. The filtrate was then added to a mixture of silver sulfate (3.68 g, 11.81 mmol) and iodine (3.00 g, 11.81 mmol) in MeOH (10 mL). The mixture was stirred at room temperature for 3 h until the color of solution became light yellow. The mixture was filtered and the filtrate was concentrated. The title compound was obtained after flash column on silica gel using EtOAc:hexane 5:95 as the elute. ¹H NMR (CDCl₃, 500 MHz) δ 7.61 (d, J=8.0 Hz, 1H), 6.56 (d, J=12.5 Hz, 1H), 3.90 (s, 3H), 3.18 (m, 1H), 1.28 (m, 6H).

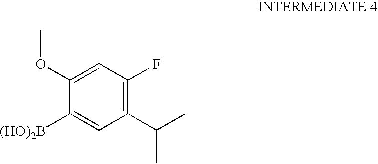

### (4-fluoro-5-isopropyl-2-methoxyphenyl)boronic acid

To a solution of 1-fluoro-4-iodo-2-isopropyl-5-methoxybenzene (Intermediate 3, 2.61 g, 8.88 mmol) in THF at −78° C., n-BuLi (4.26 mL, 10.65 mmol, 2.5 M) was added dropwise. The solution was stirred at −78° C. for 30 min. Trimethyl borate (2.98 mL, 26.6 mmol) was added. The solution was then stirred at −78° C. for 3 h. The reaction was quenched at −78° C. with saturated ammonium chloride and the mixture was warmed to room temperature. The organic layer was extracted with ethyl acetate (3×50 mL). The combined ethyl acetate layers were washed with brine and dried over sodium sulfate. The title compound was obtained as a white solid. ¹H NMR (CDCl₃, 500 MHz) δ 7.74 (d, J=10.0 Hz, 1H), 6.62 (d, J=12.5 Hz, 1H), 5.65 (br s, 2H), 3.92 (s, 3H), 3.20 (m, 1H), 1.22 (m, 6H).

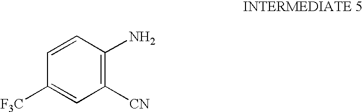

### 2-Amino-5-(trifluoromethyl)benzonitrile

A 2-liter flask was charged with 100 g (0.348 mol) of 4-amino-3-iodobenzotrifluoride, 40 g of CuCN and 750 mL of DMF. The mixture was heated to and then maintained at reflux for 1 hour. The reaction was cooled and poured into 3 L of water containing 300 mL of concentrated ammonium hydroxide. To the mixture was added 1 L CH₂Cl₂. The mixture was then filtered through Celite. The layers were separated and the aqueous layer was back extracted with CH₂Cl₂. The organic extracts were combined and the solvent removed under reduced pressure. The residue was dissolved in 1.5 L of ether and the resulting solution was washed with 1N ammonium hydroxide, aqueous sodium bisulfite, 1N aqueous HCl and brine. The solution was dried over anhydrous MgSO₄ and filtered through a silica gel plug containing a layer of MgSO₄ on top. The plug was washed with 0.5 L ether. The ether solutions were combined and concentrated to 750 mL and let stand at room temperature. After 2 days the resulting solids were collected, washed with hexanes and dried under reduced pressure to afford 2-amino-5-(trifluoromethyl)benzonitrile. ¹H NMR (CDCl₃, 500 MHz) δ 7.68 (s, 1H), 7.58 (d, J=8.5 Hz, 1H), 6.81 (d, J=8.5 Hz, 1H), 4.80 (br s, 2H).

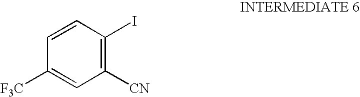

### 2-Iodo-5-(trifluoromethyl)benzonitrile

To a solution of 2-amino-5-(trifluoromethyl)benzonitrile (Intermediate 5, 15.1 g) and diiodomethane (24 mL) in acetonitrile (150 mL) at 35° C. was added t-butyl nitrite (21 mL) dropwise. The reaction was maintained at approximately 35° C. during the addition. The reaction was aged for 30 min and then heated to 60° C. for 30 minutes. The reaction mixture was cooled, diluted with ether and washed twice with water, twice with aqueous sodium bisulfite, water and then brine. The solution was dried over anhydrous MgSO₄, filtered through a silica gel plug and then concentrated giving afford a red oil. The product was purified by silica gel chromatography eluting sequentially with hexanes, 3:1 hexanes/CH₂Cl₂ and 1:1 hexanes/CH₂Cl₂ to afford 2-iodo-5-(trifluoromethyl)benzonitrile. ¹H NMR (CDCl₃, 500 MHz) δ 8.10 (d, J=8.5 Hz, 1H), 7.85 (d, J=1.8 Hz, 1H), 7.52 (dd, J=8.5, 1.8 Hz, 1H).

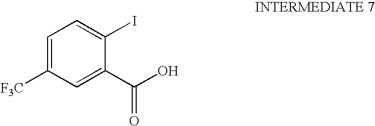

### 2-Iodo-5-(trifluoromethyl)benzoic acid

Potassium hydroxide (3.78 g; 0.0673 mol) was added to a stirred solution of 2-iodo-5-(trifluoromethyl)benzonitrile (Intermediate 6; 4 g; 0.0135 mol) in a 1:1 isopropanol:H₂O solution (60 mL). The reaction was heated at reflux for 14 h and then partitioned between H₂O (50 mL) and EtOAc (50 mL). The aqueous layer was extracted with EtOAc (50 mL) and acidified to pH 5 with 6N HCl. The aqueous layer was further extracted with EtOAc (4×50 mL) and the combined extracts were washed with brine (50 mL), dried over MgSO₄, filtered, and concentrated in vacuo to afford 2-iodo-5-(trifluoromethyl)benzoic acid as a yellow solid. LCMS=317.0 (M+1)⁺. ¹H NMR (CDCl₃, 500 MHz): δ 8.27 (d, J=1.6 Hz, 1H), 8.25 (d, J=8.2 Hz, 1H), 7.47 (dd, J=8.2, 1.8 Hz, 1H).

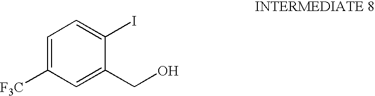

### [2-Iodo-5-(trifluoromethyl)phenyl]methanol

Borane-THF (1.0M solution in THF; 94 mL; 94 mmol) was added to a stirred solution of 2-iodo-5-(trifluoromethyl)benzoic acid (Intermediate 7, 2.97 g; 9.4 mmol) in THF (300 mL) at 0° C. under N₂. The reaction was heated at reflux for 90 min and then carefully quenched with 6N HCl until no further gas evolution. The reaction was diluted with H₂O (250 mL) and extracted with EtOAc (3×250 mL). The combined extracts were washed with brine (300 mL), dried over MgSO₄, filtered, and concentrated in vacuo. The crude material was purified by flash chromatography on silica gel (0-25% EtOAc/hexanes gradient) to afford [2-iodo-5-(trifluoromethyl)phenyl]methanol as a white solid. LCMS=285.0 (M 17)⁺. ¹H NMR (CDCl₃, 500 MHz): δ 7.97 (d, J=8.3 Hz, 1H), 7.79 (s, 1H), 7.28 (d, J=8.4 Hz, 1H), 4.75 (s, 2H).

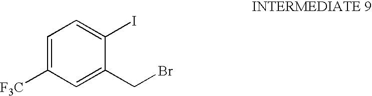

### 2-(Bromomethyl)-1-iodo-4-(trifluoromethyl)benzene

Carbon tetrabromide (1.86 g; 5.6 mmol) and triphenylphosphine (1.47 g; 5.6 mmol) were added successively to a stirred solution of [2-iodo-5-(trifluoromethyl)phenyl]methanol (Intermediate 8, 1.13 g; 3.74 mmol) in CH₂Cl₂ (25 mL) at 0° C. under N₂. The reaction was stirred at room temperature for 48 h. A second equivalent of carbon tetrabromide (1.2 g; 3.74 mmol) and triphenylphosphine (0.98 g; 3.74 mmol) was added and the reaction was stirred an additional 14 h. The solvent was removed in vacuo and the residue was purified by flash chromatography on silica gel (0-25% EtOAc/hexanes gradient) to afford 2-(bromomethyl)-1-iodo-4-(trifluoromethyl)benzene as a clear oil. ¹H NMR (CDCl₃, 500 MHz): δ 8.02 (d, J=8.2 Hz, 1H), 7.73 (d, J=1.8 Hz, 1H), 7.26 (dd, J=8.3, 1.8 Hz, 1H), 4.64 (s, 2H).

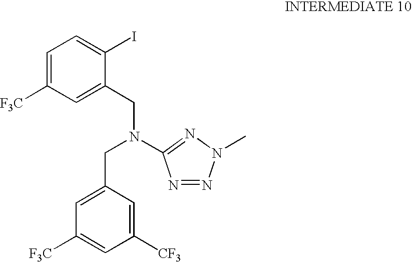

### N-[3,5-bis trifluoromethyl)benzyl]-N-[2-iodo-5-trifluoromethyl)benzyl]-2-methyl-2H-tetrazol-5-amine

To a stirred suspension of sodium hydride (60% in oil; 30.7 mg, 0.77 mmol) in THF (1 mL) at 0° C. under an atmosphere of N₂ was added a solution of N-[3,5-bis(trifluoroethyl)benzyl]-2-methyl-2H-tetrazol-5-amine (Intermediate 1; 100 mg, 0.31 mmol) in THF (2 mL) dropwise. The resultant mixture stirred at 0° C. for 20 min prior to addition of 2-(bromomethyl)-1-iodo-4-(trifluoromethyl)benzene (Intermediate 9; 135 mg, 0.37 mmol) as a solution in THF (1 mL). The reaction was allowed to warm to room temperature and stirred for 5 h. The reaction was quenched with H₂O and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-25% EtOAc/hexanes gradient) to afford N-[3,5-bis(trifluoromethyl)benzyl]-N-[2-iodo-5-(trifluoromethyl)benzyl]-2-methyl-2H-tetrazol-5-amine as a clear oil. LCMS=610.0 (M+1)⁺. ¹H NMR (CDCl₃, 500 MHz): δ 7.98 (d, J=8.2 Hz, 1H), 7.79 (s, 1H), 7.70 (s, 2H), 7.42 (s, 1H), 7.24 (d, J=8.2 Hz, 1H), 4.83 (s, 2H), 4.81 (s, 2H), 4.26 (s, 3H).

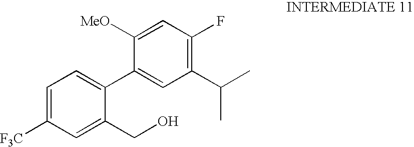

### [4′-fluoro-5′-isopropyl-2′-methoxy-4-(trifluoromethyl)biphenyl-2-yl]methanol

A mixture of [2-iodo-5-(trifluoromethyl)phenyl]methanol (Intermediate 8, 3.09 g, 10.2 mmol), (4-fluoro-5-isopropyl-2-methoxyphenyl)boronic acid (Intermediate 4, 4.34 g, 20.5 mmol), (Ph₃P)₄Pd (1.42 g, 1.23 mmol) and Na₂CO₃ (9.11 g, 85.9 mmol) in benzene/EtOH/H₂O (7:1:3, 250 mL) was heated at reflux for 24 h under N₂. After cooling to room temperature, the aqueous phase was separated and extracted with CH₂Cl₂ (3×50 mL). The combined organic layers were dried (Na₂SO₄) and concentrated in vacuo to give the crude product. This was purified by flash chromatography on silica gel (65×200 mm, 0-20% EtOAc in hexanes gradient) to afford 4′-fluoro-5′-isopropyl-2′-methoxy-4-(trifluoromethyl)biphenyl-2-yl]methanol. R_{f}=0.50 (20% EtOAc in hexanes). ¹H NMR (500 MHz, CDCl₃) δ 7.86 (s, 1H), 7.59 (d, J=6.7 Hz, 1H), 7.30 (d, J=7.9 Hz, 1H), 6.99 (d, J=8.6 Hz, 1H), 6.68 (d, J=12.0 Hz, 1H), 4.52 (br s, 1H), 4.46 (br s, 1H), 3.73 (s, 3H), 3.25-3.17 (m, 1H), 1.82 (br s, 1H), 1.24 (d, J=6.8 Hz, 6H).

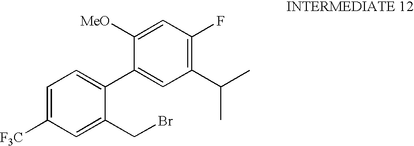

### 2′-(bromomethyl)-4-fluoro-5-isopropyl-2-methoxy-4′-(trifluoromethyl)biphenyl

A solution of triphenylphosphine (3.11 g, 11.8 mmol) in dry CH₂Cl₂ (7 mL) was added by cannula to a stirred solution of carbon tetrabromide (3.93 g, 11.8 mmol) and 4′-fluoro-5′-isopropyl-2′-methoxy-4-(trifluoromethyl)biphenyl-2-yl)methanol (Intermediate 11, 3.38 g, 9.87 mmol) in dry CH₂Cl₂ (56 mL) at 0° C. under N₂. The reaction was allowed to warm to room temperature. After 2 h, the reaction mixture was concentrated in vacuo to give the crude product. This was purified by flash chromatography on silica gel (65×200 mm, 0-20% EtOAc in hexanes gradient) to afford 2′-(bromomethyl)-4-fluoro-5-isopropyl-2-methoxy-4′-(trifluoromethyl)biphenyl. ¹H NMR (500 MHz, CDCl₃) δ 7.83 (s, 1H), 7.61 (d, J=8.0 Hz, 1H), 7.35 (d, J=8.0 Hz, 1H), 7.15 (d, J=8.6 Hz, 1H), 6.72 (d, J=12.0 Hz, 1H), 4.43 (br d, J=10.0 Hz, 1H), 4.30 (br d, J=10.2 Hz, 1H), 3.76 (s, 3H), 3.30-3.22 (m, 1H), 1.29 (d, J=6.9 Hz, 6H).

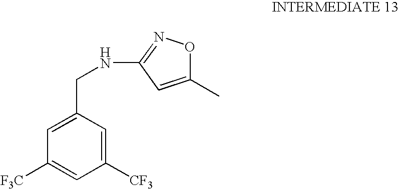

### N-[3,5-bis(trifluoromethyl)benzyl]-5-methylisoxazol-3-amine

A stirred solution of 3,5-bis(trifluoromethyl)benzaldehyde (338 μL, 2.04 mmol) and 5-methylisoxazol-3-amine (200 mg, 2.04 mmol) in toluene (8 mL) was heated at reflux for 3.5 h. The reaction was concentrated in vacuo and the residue was redissolved in EtOH (8 mL). Sodium borohydride (154 mg, 4.08 mmol) was added and the mixture was stirred at room temperature for 14 h. The reaction was quenched with sat. NH₄Cl and was partitioned between H₂O (25 mL) and EtOAC (25 mL). The aqueous layer was re-extracted with EtOAc (3×25 mL) and the combined extracts were washed with brine (50 mL), dried (Na₂SO₄), filtered and concentrated in vacuo. The residue was purified by flash chromatography on silica gel (65×200 mm, 0-25% EtOAc in hexanes gradient) to give N-[3,5-bis(trifluoromethyl)benzyl]-5-methylisoxazol-3-amine as a white solid. LCMS=325.1 (M+1)⁺.

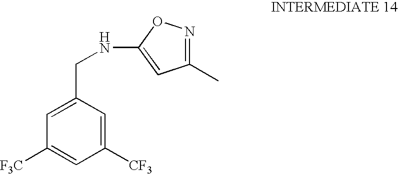

### N-[3,5-bis(trifluoromethyl)benzyl]-3-methylisoxazol-5-amine

A stirred solution of 3,5-bis(trifluoromethyl)benzaldehyde (338 μL, 2.04 mmol) and 3-methylisoxazol-5-amine (200 mg, 2.04 mmol) in toluene (8 mL) was heated at reflux for 3.5 h. The reaction was concentrated in vacuo and the residue was redissolved in EtOH (8 mL). Sodium borohydride (154 mg, 4.08 mmol) was added and the mixture was stirred at room temperature for 14 h. The reaction was quenched with sat. NH₄Cl and was partitioned between H₂O (25 mL) and EtOAC (25 mL). The aqueous layer was re-extracted with EtOAc (3×25 mL) and the combined extracts were washed with brine (50 mL), dried (Na₂SO₄), filtered and concentrated in vacuo. The residue was purified by flash chromatography on silica gel (65×200 mm, 0-25% EtOAc in hexanes gradient) to afford N-[3,5-bis(trifluoromethyl)benzyl]-3-methylisoxazol-5-amine as a white solid. LCMS=325.1 (M-4-1)⁺.

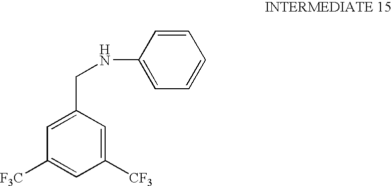

### N-[3,5-bis(trifluoromethyl)benzyl]aniline

A stirred solution of 3,5-bis(trifluoromethyl)benzaldehyde (356 μL, 2.15 mmol) and aniline (200 mg, 2.15 mmol) in toluene (8 mL) was heated at reflux for 3.5 h. The reaction was concentrated in vacuo and the residue was redissolved in EtOH (8 mL). Sodium borohydride (163 mg, 4.30 mmol) was added and the mixture was stirred at room temperature for 14 h. The reaction was quenched with sat. NH₄Cl and was partitioned between H₂O (25 mL) and EtOAC (25 mL). The aqueous layer was re-extracted with EtOAc (3×25 mL) and the combined extracts were washed with brine (50 mL), dried (Na₂SO₄), filtered and concentrated in vacuo. The residue was purified by flash chromatography on silica gel (0-25% EtOAc in hexanes gradient) to afford N-[3,5-bis(trifluoromethyl)benzyl]aniline as a yellow oil. LCMS=320.2 (M+1)⁺.

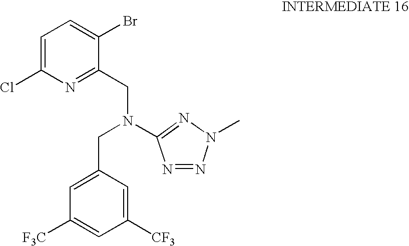

### N-[3,5-bis(trifluoromethyl)benzyl]-N-[3-bromo-6-chloropyridin-2-yl)methyl]-2-methyl-2H-tetrazol-5-amine

To a stirred suspension of sodium hydride (60% in oil; 1-C5 mg, 2.53 mmol) in THF (10 mL) at 0° C. under an atmosphere of N₂ was added a solution of N-[3,5-bis(trifluoromethyl)benzyl]-2-methyl-2H-tetrazol-5-amine (Intermediate 1; 411 mg, 1.26 mmol) in THF (2 mL) dropwise. The resultant mixture was stirred at 0° C. for 20 min prior to the addition of 3-bromo-2-bromomethyl-6-chloropyridine (300 mg, 1.05 mmol) as a solution in THF (3 mL). The reaction was allowed to warm to room temperature and stirred for 14 h. The reaction was quenched with H₂O and was partitioned between H₂O (25 mL) and EtOAC (35 mL). The aqueous layer was re-extracted with EtOAc (3×35 mL) and the combined extracts were washed with brine (25 mL), dried (MgSO₄), filtered and concentrated in vacuo. The residue was purified by flash silica gel chromatography (0-25% EtOAc/hexanes gradient) to afford N-[3,5-bis(trifluoromethyl)benzyl]-N-[(3-bromo-6-chloropyridin-2-yl)methyl]-2-methyl-2H-tetrazol-5-amine as a clear oil. LCMS=531 (M+1)⁺. ¹H NMR (CDCl₃, 500 MHz): δ 7.76-7.75 (brs, 3H), 7.70 (d, J=8.2 Hz, 1H), 7.07 (d, J=8.2 Hz, 1H), 4.91 (brs, 2H),), 4.89 (brs, 2H), 4.19 (s, 3H).

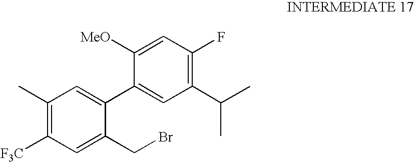

### 2-(bromomethyl)-4′-fluoro-5′-isopropyl-2′-methoxy-5-methyl-4-(trifluoromethyl)biphenyl

### Step A: 2-iodo-4-methyl-5-(trifluoromethyl)aniline

4-Methyl-3-(trifluoromethyl)aniline (100 mg, 0.571 mmol) was added dropwise to a stirred suspension of silver sulfate (178 mg, 0.571 mmol) in a solution of iodine (145 mg, 0.571 mmol) in EtOH (4.5 mL) at room temperature under N₂. The mixture was stirred overnight and filtered through a plug of Celite. The filtrate was concentrated in vacuo to afford 2-iodo-4-methyl-5-(trifluoromethyl)aniline. LCMS calc.=302.0; found=302.1 (M+1)⁺. ¹H NMR (500 MHz, CD₃OD): δ 7.79 (s, 1H); 7.39 (s, 1 I); 5.25 (br s, 2H); 2.34 (s, 3H).

### Step B: 4′-fluoro-5′-isopropyl-2′-methoxy-5-methyl-4-(trifluoromethyl)biphenyl-2-amine

A mixture of 2-iodo-4-methyl-5-(trifluoromethyl)aniline (Step A, 200.9 mg, 0.667 mmol), (4-fluoro-5-isopropyl-2-methoxyphenyl)boronic acid (Intermediate 4, 212 mg, 1.00 mmol) and 1,1′-bis(di-t-butylphosphino)ferrocene palladium dichloride (43.5 mg, 0.0667 mmol) in 1N aqueous K₂CO₃ (6.7 mL) and THF (6.7 mL) was degassed and heated at reflux under N₂ for 3 h. The reaction mixture was cooled, diluted with water (10 mL) and extracted with EtOAc (3×20 mL). The combined extracts were dried (Na₂SO₄) and concentrated in vacuo to give the crude product. This was purified by flash chromatography on silica gel (25×160 mm, 0-40% EtOAc in hexanes gradient) to afford 4′-fluoro-5′-isopropyl-2′-methoxy-5-methyl-4-(trifluoromethyl)biphenyl-2-amine as a colorless solid. R_{f}=0.54 (20% EtOAc/hexanes). LCMS calc.=342.2; found=342.1 (M+1)⁺. ¹H NMR (500 MHz, CDCl₃): δ 7.10 (d, J=8.7 Hz, 1H); 7.02 (s, 1H); 6.99 (s, 1H); 6.71 (d, J-=12.1 Hz, 1H); 3.80 (s, 3H); 3.71 (s, 2H); 3.26-3.18 (m, 1H); 2.40 (d, J=1.6 Hz, 3H); 1.27 (d, J=6.8 Hz, 6 II).

### Step C: 4-fluoro-2′-iodo-5-isopropyl-2-methoxy-5′-methyl-4′-(trifluoromethyl)biphenyl

Isoamyl nitrite (96%, 51 μL, 43.2 mg, 0.369 mmol) was added to a solution of 4′-fluoro-5′-isopropyl-2′-methoxy-5-methyl-4-(trifluoromethyl)biphenyl-2-amine (Step B, 84.0 mg, 0.246 mmol) and iodine (68.7 mg, 0.271 mmol) at room temperature under N₂. The solution was stirred for 5 min then heated at reflux for 2 h. The reaction mixture was diluted with EtOAc (20 mL), washed with saturated Na₂SO₃ (10 mL), saturated NaHCO₃ (10 mL) and brine (10 mL), then dried (Na₂SO₄) and concentrated in vacuo to give the crude product. This was purified by flash chromatography on silica gel (25×160 mm, 0-10% EtOAc in hexanes gradient) to afford 4-fluoro-2′-iodo-5-isopropyl-2-methoxy-5′-methyl-4′-(trifluoromethyl)biphenyl (72 mg). R_{f}=0.85 (10% EtOAc/hexanes). ¹H NMR (500 MHz, CDCl₃): δ 8.18 (s, 1H); 7.03 (d, J=8.5 Hz, 1H); 6.74 (d, J=11.9 Hz, 1H); 3.82 (s, 3H); 3.33-3.25 (m, 1H); 2.51 (s, 3H); 1.35-1.31 (m, 6H).

### Step D: methyl 4′-fluoro-5′-isopropyl-2′-methoxy-5-methyl-4-(trifluoromethyl)biphenyl-2-carboxylate

A solution of 4-fluoro-2′-iodo-5-isopropyl-2-methoxy-5′-methyl-4′-(trifluoromethyl)biphenyl (Step C, 71.9 mg, 0.159 mmol), propane-1,3-diylbis(diphenylphosphine) (17.0 mg, 0.0413 mmol), triethylamine (119 mg, 163 μL, 1.17 mmol) and palladium (II) acetate (6.4 mg, 0.0286 mmol) in DMF/MeOH (1:1, 3 mL) was heated at 70° C. under CO (60 psi) for 18 h. The reaction mixture was cooled and 50% saturated brine (20 mL) was added. The mixture was extracted with EtOAc (3×20 mL). The combined extracts were dried (Na₂SO₄) and concentrated in vacuo to give the crude product. This was purified by flash chromatography on silica gel (25×160 mm, 0-20% EtOAc in hexanes gradient) to afford methyl 4′-fluoro-5′-isopropyl-2′-methoxy-5-methyl-4-(trifluoromethyl)biphenyl-2-carboxylate. R_{f}=0.75 (20% EtOAc/hexanes). LCMS calc.=353.1; found=353.1 (M+1)^{÷}. ¹H NMR (600 MHz, CDCl₃): 8.14 (s, 1H); 7.25 (s, 1H); 7.08 (d, J=8.5 Hz, 1H); 6.62 (d, J=12.0 Hz, 1H); 3.71 (s, 3H); 3.70 (s, 3H); 3.25-3.19 (m, 1H); 2.56 (s, 3H); 1.29 (d, J=7.0 Hz, 6H).

### Step E: [4′-fluoro-5′-isopropyl-2′-methoxy-5-methyl-4-(trifluoromethyl)biphenyl-2-yl]methanol

A solution of lithium borohydride in THF (461 μL, 0.461 mmol) was added dropwise to a stirred solution of methyl 4′-fluoro-5′-isopropyl-2′-methoxy-5-methyl-4-(trifluoromethyl)biphenyl-2-carboxylate (Step D, 35.4 mg, 0.0921 mmol) in dry THF (4 mL) at room temperature under N₂. After 4 h the reaction mixture was diluted with 1N HCl (5 mL) and water (10 mL) then extracted with EtOAc (3×20 mL). The combined extracts were dried (Na₂SO₄) and concentrated in vacuo to give the crude product. This was purified by flash chromatography on silica gel (25×160 mm, 0-20% EtOAc in hexanes gradient) to afford [4′-fluoro-5′-isopropyl-2′-methoxy-5-methyl-4-(trifluoromethyl)biphenyl-2-yl]methanol. R_{f}=0.51 (20% EtOAc/hexanes). NMR (500 MHz, CDCl₃): δ 7.82 (s, 1H); 7.12 (s, 1H); 7.01 (d, J=8.5 Hz, 1H); 6.69 (d, J=12.0 Hz, 1H); 4.46-4.41 (m, 2H); 3.74 (s, 3H); 3.26-3.18 (m, 1H); 2.51 (s, 3H); 2.12 (br s, 1H); 1.27 (br d, J=5.1 Hz, 6H).

### Step F: 2-(bromomethyl)-4′-fluoro-5′-isopropyl-2′-methoxy-5-methyl-4-(trifluoromethyl)biphenyl

Triphenylphosphine (29.5 mg, 0.112 mmol) was added to a stirred solution of [4′-fluoro-5′-isopropyl-2′-methoxy-5-methyl-4-(trifluoromethyl)biphenyl-2-yl]methanol (Step E, 33.4 mg, 0.0937 mmol) and carbon tetrabromide (37.3 mg, 0.112 mmol) in dry CH₂Cl₂ (1 mL) at 0° C. under N₂. The reaction was allowed to warm to room temperature overnight. The reaction mixture was concentrated in vacuo to afford the crude product. This was purified by flash chromatography on silica gel (25×160 mm, 0-20% EtOAc in hexanes gradient) to afford 2-(bromomethyl)-4′-fluoro-5′-isopropyl-2′-methoxy-5-methyl-4-(trifluoromethyl)biphenyl. R_{f}=0.92 (20% EtOAc/hexanes). ¹H NMR (500 MHz, CDCl₃): δ 7.81 (s, 1 H); 7.18 (s, 1H); 7.17 (d, J=8.5 Hz, 1H); 6.73 (d, J=12.0 Hz, 1H); 4.41 (d, J=10.1 Hz, 1H); 4.29 (d, J=10.1 Hz, 1H); 3.77 (s, 3H); 3.32-3.24 (m, 1H); 2.53 (s, 3H); 1.31 (d, J=6.9 Hz, 8H).

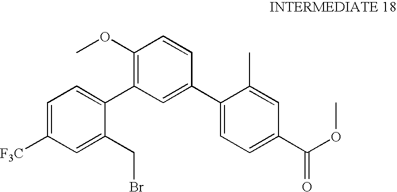

### methyl 2″-(bromomethyl)-4′-methoxy-2-methyl-4″-(trifluoromethyl)-1,1′:3′,1″-terphenyl-4-carboxylate

### Step 1: methyl 4′-methoxy-2-methylbiphenyl-4-carboxylate

To methyl 4-bromo-3-methyl benzoate (92 g, 0.402 mol), (4-methoxyphenyl)boronic acid (61.1 g, 0.402 mol), Na₂CO₃ (85.2 g, 0.804 mol), and PdCl₂(PPh₃)₂ (1410 mg, 2.01 mmol) was added EtOH (1.23 L) and water (0.61 L). The reaction was then heated to 80° C. for 1 hour. The reaction was cooled to room temperature, 550 ml of water was added, and the mixture was left standing for 1 hour. The resulting solids were filtered and washed with a solution of EtOH and H₂O (1:1, 750 mL). The solids were ground using a mortar and pestle, then were slurried in 250 mL H₂O at room temperature for 1 h, then were filtered and washed with water (2×125 mL), and were dried to give methyl 4′-methoxy-2-methylbiphenyl-4-carboxylate. 1H NMR (CDCl₃, 400 MHz) δ 7.95 (s, 1H), 7.89 (d, J=7.9 Hz, 1H), 7.29 (d, J=7.9 Hz, 1H), 7.27 (d, J=8.7 Hz, 2H), 6.98 (d, J=8.7 Hz, 2H), 3.94 (s, 3H), 3.87 (s, 3H), 2.33 (s, 3H).

### Step 2: methyl 3′-bromo-4′-methoxy-2-methylbiphenyl-4-carboxylate

To a solution of methyl 4′-methoxy-2-methylbiphenyl-4-carboxylate (71.5 g, 0.279 mol) in acetonitrile (1.43 L) and water (572 mL) was added oxone (180.1 g, 0.293 mol). Then a solution of KBr (38.2 g, 0.321 mol) in water (143 mL) was slowly added over 30 minutes. The reaction was stirred for 2.5 hours, then water (715 mL) was added, and the mixture was left standing for 1 hour. The solids were filtered and washed as follows: with a solution of MeCN/water (1:1, 350 mL, twice), then water (700 mL, twice, then 350 mL), and then were dried to afford methyl 3′-bromo-4′-methoxy-2-methylbiphenyl-4-carboxylate. 1H NMR (CDCl₃, 400 MHz) δ 7.94 (s, 1H), 7.89 (d, J=8.1 Hz, 1H), 7.53 (d, J=2.2 Hz, 1H), 7.3-7.2 (m, 2H), 6.97 (d, J=8.4 Hz, 1H), 3.96 (s, 3H), 3.94 (s, 3H), 2.32 (s, 3H).

### Step 3: methyl 2″-(hydroxymethyl)-4′-methoxy-2-methyl-4″-(trifluoromethyl)-1,1′:3′,1″-terphenyl-4-carboxylate

To a mixture of methyl 3′-bromo-4′-methoxy-2-methylbiphenyl-4-carboxylate (80.0 g, 0.239 mol), pinacole borane (72.8 g, 0.287 mol), Pd(dba)₂ (4120 mg, 7.17 mmol), P(Cy)₃ (2140 mg, 7.65 mmol), and KOAc (70.3 g, 0.717 mol) was added dioxane (1.2 L). The reaction was heated to 80° C. and stirred for 3 hours. The reaction was then cooled to room temperature and filtered. The solids were dissolved in EtOAc (800 mL), washed with brine (400 mL, twice), and concentrated. The residue was dissolved in THF (300 mL), and [2-chloro-5-(trifluoromethyl)phenyl]methanol (47.1 g, 0.223 mol) and (t-Bu₂P)₂ferrocene PdCl₂ were added. A solution of K₂CO₃ (83.7 g, 0.606 mol) in water (214 mL) was added, and the mixture was heated to 45° C. and stirred for 9 hours. The reaction was cooled to room temperature, diluted with EtOAc (428 mL), and washed with water (428 mL) and brine (428 mL). To the organic material was added 21.5 g charcoal (Darco KB-100 mesh), and the mixture was stirred for 1 hour. The mixture was filtered, and the solid material was washed with EtOAc (428 mL). The filtrate was concentrated and then re-dissolved in MeOH (677 mL) and left to stand for 1 hour. To the mixture was added water (169 mL) over 2 hours, and then the mixture was left to stand for 1 hour. The resulting solids were washed with a solution of MeOH and water (4:1, 170 mL, three times) and dried to afford methyl 2″-(hydroxymethyl)-4′-methoxy-2-methyl-4″-(trifluoromethyl)-1,1′:3′,1″-terphenyl-4-carboxylate.

### Step 4: methyl 2″-(bromomethyl)-4′-methoxy-2-methyl-4″-(trifluoromethyl)-1,1′:3′,1″-terphenyl-4-carboxylate

To a 0° C. solution of methyl 2″-(hydroxymethyl)-4′-methoxy-2-methyl-4″-(trifluoromethyl)-1,1′:3′,1″-terphenyl-4-carboxylate (1.500 g, 3.49 mmol) in CH₂Cl₂ (14 mL) was added CBr₄ (2.429 g, 7.33 mmol). Then a solution of triphenyl phosphine (1.830 g, 6.98 mmol) in CH₂Cl₂ (15 mL) was added. The solution was warmed to room temperature and stirred for twelve hours. The reaction was concentrated, and the residue was purified by flash chromatography on silica gel (0 to 25% EtOAc/hexanes) to afford methyl 2″-(bromomethyl)-4′-methoxy-2-methyl-4″-(trifluoromethyl)-1,1′:3′,1″-terphenyl-4-carboxylate. R_{f}=0.59 (50% EtOAc/hexanes). LCMS=494.8 (M+1)+. 1H NMR (CDCl₃, 500 MHz) δ 7.95 (s, 1H), 7.89 (d, J=8.0 Hz, 1H), 7.80 (s, 1H), 7.59 (d, J=7.6 Hz, 1H), 7.40-7.33 (m, 3H), 7.21 (d, J=2.3 Hz, 1H), 7.06 (d, J=8.5 Hz, 1H), 4.44-4.39 (m, 2H), 3.93 (s, 3H), 3.82 (s, 3H), 2.37 (s, 3H).

### Example 1

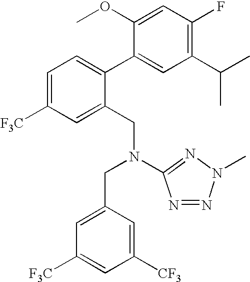

### N-[3,5-bis(trifluoromethyl)benzyl]-N-{[4′-fluoro-5′-isopropyl-2′-methoxy-4-(trifluoromethyl)biphenyl-2-yl]methyl}-2-methyl-2H-tetrazol-5-amine

To a stirred suspension of sodium hydride (60% in oil; 12 mg, 0.31 mmol) in THF (1 mL) at 0° C. under an atmosphere of N₂ was added N-[3,5-bis(trifluoromethyl)benzyl]-2-methyl-2H-tetrazol-5-amine (Intermediate 1; 48 mg, 0.15 mmol) portionwise. The resultant solution was stirred at 0° C. for 20 min prior to the addition of a solution of 2′-(bromomethyl)-4-fluoro-5-isopropyl-2-methoxy-4′-(trifluoromethyl)biphenyl (Intermediate 12; 50 mg, 0.12 mmol) in THF (1 mL). The reaction was allowed to warm to room temperature and stirred for 14 h. The reaction was quenched with H₂O and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-10% EtOAc/hexanes gradient) to afford N-[3,5-bis(trifluoromethyl)benzyl]-N-{[4′-fluoro-5′-isopropyl-2′-methoxy-4-(trifluoromethyl)biphenyl-2-yl]methyl}-2-methyl-2H-tetrazol-5-amine as a yellow gum. LCMS=650.2 (M+1)⁺. ¹H NMR (CDCl₃, 500 MHz): δ 7.73 (s, 1H), 7.58 (d, J=8.0 Hz, 1H), 7.51 (s, 2H), 7.50 (s, 1H), 7.32 (d, J=8.0 Hz, 1H), 6.92 (d, J=8.5 Hz, 1H), 6.60 (d, J=12.1 Hz, 1H), 4.63 (s, 2H), 4.51 (s, 2H), 4.16 (s, 3H), 3.70 (s, 3H), 3.81-3.13 (m, 1H), 1.23 (d, J=7.1 Hz, 3H), 1.19 (d, J=6.9 Hz, 3H).

### Example 2

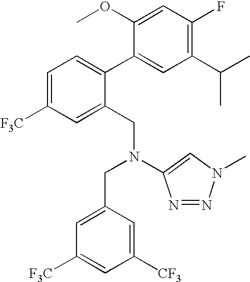

### N-[3,5-bis(trifluoromethyl)benzyl]-N-{[4′-fluoro-5′-isopropyl-2′-methoxy-4-(trifluoromethyl)biphenyl-2-yl]methyl}-1-methyl-1H-1,2,3-triazol-4-amine

### Step A: N-[3,5-bis(trifluoromethyl)benzyl]-1-methyl-1H-1,2,3-triazol-4-amine

3,5-bis(trifluoromethyl)benzaldehyde (42 μL, 0.25 mmol) was treated with 1-methyl-1H-1,2,3-triazol-4-amine (25 mg, 0.25 mmol) followed by sodium borohydride (19 mg, 0.51 mmol) as described in Intermediate 1. The residue was purified by flash silica gel chromatography (0-75% EtOAc/hexanes gradient) to afford N-[3,5-bis(trifluoromethyl)benzyl]-1-methyl-1H-1,2,3-triazol-4-amine as a white solid. LCMS=325.2 (M+1)⁺. ¹H NMR (CDCl₃, 500 MHz): δ 7.85 (s, 2 II), 7.79 (s, 1 II), 6.68 (s, 1 II), 4.48 (s, 2H), 3.97 (s, 3H).

### Step B: N-[3,5-bis(trifluoromethyl)benzyl]-N-{[4′-fluoro-5′-isopropyl-2′-methoxy-4-(trifluoromethyl)biphenyl-2-yl]methyl}-1-methyl-1H-1,2,3-triazol-4-amine

N-[3,5-bis(trifluoromethyl)benzyl]-1-methyl-1H-1,2,3-triazol-4-amine (Step A; 44 mg, 0.14 mmol) was treated with sodium hydride (60% in oil; 12.3 mg, 0.31 mmol) and 2′-(bromomethyl)-4-fluoro-5-isopropyl-2-methoxy-4′-(trifluoromethyl)biphenyl (Intermediate 12; 50 mg, 0.12 mmol) as described in Example 1 to afford N-[3,5-bis(trifluoromethyl)benzyl]-N-{[4′-fluoro-5′-isopropyl-2′-methoxy-4-(trifluoromethyl)biphenyl-2-yl]methyl}-1-methyl-1H-1,2,3-triazol-4-amine as a clear oil. LCMS=649.3 (M+1)⁺. ¹H NMR (CDCl₃, 500 MHz): δ 7.72 (s, 1H), 7.64 (s, 2H), 7.57 (s, 1H), 7.54 (d, J=8.0 Hz, 1 H), 7.28 (d, J=7.8 Hz, 1H), 6.90 (d, J=8.5 Hz, 1H), 6.62 (d, J=12.1 Hz, 1H), 6.39 (s, 1H), 4.57 (d, J=7.3 Hz, 2H), 4.36-4.17 (m, 2H), 3.92 (s, 3H), 3.67 (s, 3H), 3.20-3.14 (m, 1H), 1.26-1.17 (m, 6H).

### Example 3

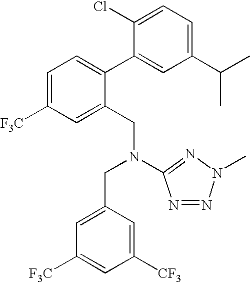

### N-[3,5-bis(trifluoromethyl)benzyl]-N-{[2′-chloro-5′-isopropyl-4-(trifluoromethyl)biphenyl-2-yl]methyl}-2-methyl-2H-tetrazol-5-amine

To a solution of N-[3,5-bis(trifluoromethyl)benzyl]-N-[2-iodo-5-(trifluoromethyl)benzyl]-2-methyl-2H-tetrazol-5-amine (Intermediate 10; 37 mg, 0.06 mmol)

and (2-chloro-5-isopropylphenyl)boronic acid (18 mg, 0.91 mmol) in THF (1.0 mL) was added aqueous 1M K₂CO₃ (1.0 mL) and the solution was degassed with nitrogen for 2 minutes. 1,1-bis(di-t-butylphosphine)ferrocene palladium dichloride (7.8 mg, 0.12 mmol) was added and the solution was heated under reflux for 14 h. The reaction was cooled to room temperature, poured into H₂O (5 mL), and extracted with EtOAc (3×15 mL). The organic layers were combined and washed with brine (25 mL), dried over Na₂SO₄, filtered, and concentrated. Purification by flash chromatography on silica gel eluting with 15% EtOAC/hexanes afforded N-[3,5-bis(trifluoromethyl)benzyl]-N-{[2′-chloro-5′-isopropyl-4-(trifluoromethyl)biphenyl-2-yl]methyl}-2-methyl-2H-tetrazol-5-amine as a light yellow oil. R_{f}=0.65 (15% EtOAc/hexanes). ¹H NMR (CDCl₃, 500 MHz) δ 7.79 (s, 1H), 7.64-7.60 (m, 2H), 7.58 (s, 2H), 7.39 (m, 2H), 7.18 (dd, J=8.3 and 2.3 Hz, 1H), 6.97 (d, J=2.3 Hz, 1H), 4.67 (d, J=16 Hz, 1H), 4.62 (d, J=16 Hz, 1H), 4.54 (d, J=16 Hz, 2H), 4.16 (s, 3H), 2.93 (m, 1H), 1.21 (d, J=2.0 Hz, 3H), 1.20 (d, J=2.0 Hz, 3H).

Following the procedures outlined in EXAMPLE 3 the compounds listed in Table 1 were prepared:

TABLE 1

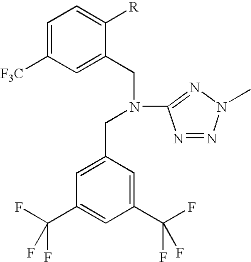

| | | LC/MS Data |
|---|---|---|
| EXAMPLE | R | (M + 1) |
| 4 | 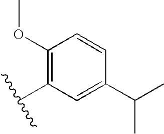 | 632.3 |
| 5 | 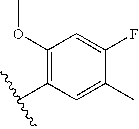 | 622.2 |
| 6 | 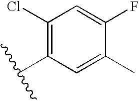 | 626.2 |

### Example 7

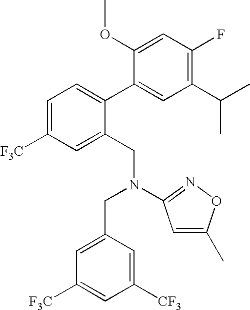

### N-[3,5-bis(trifluoromethyl)benzyl]-N-{[4′-fluoro-5′-isopropyl-2′-methoxy-4-(trifluoromethyl)biphenyl-2-yl]methyl}-5-methylisoxazol-3-amine

To a stirred suspension of potassium tert-butoxide (15 mg, 0.130 mmol) in DMF (1 mL) at 0° C. under an atmosphere of N₂ was added N-[3,5-bis(trifluoromethyl)benzyl]-5-methylisoxazol-3-amine (Intermediate 13; 30 mg, 0.093 mmol) portionwise over 5 min. The resultant solution was stirred at 0° C. for 20 min prior to the addition of a solution of 2′-(bromomethyl)-4-fluoro-5-isopropyl-2-methoxy-4′-(trifluoromethyl)biphenyl (Intermediate 12; 37 mg, 0.093 mmol) in DMF (1 mL). The reaction was allowed to warm to room temperature and stirred for 14 h. The reaction was quenched with H₂O and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-10% EtOAc/hexanes gradient) to afford N-[3,5-bis(trifluoromethyl)benzyl]-N-{[4′-fluoro-5′-isopropyl-2′-methoxy-4-(trifluoromethyl)biphenyl-2-yl]methyl}-5-methylisoxazol-3-amine as a colorless oil. LCMS=649.2 (M+1)⁺. ¹H NMR (CDCl₃, 500 MHz): δ 7.77 (s, 1H), 7.62-7/54 (m, 3H), 7.52 (s, 1H), 7.35 (d. J=8.0 Hz, 1H), 6.95 (d, J=8.5 Hz, 1H), 6.67 (d, J=11.9 Hz, 1H), 5.38 (s, 1H), 4.57-4.18 (m, 4H), 3.71 (s, 3H), 3.22 (m, 1H), 2.32 (s, 3H), 1.27 (d, J=6.9 Hz, 3H), 1.22 (d, J=6.9 Hz, 3H).

### Example 8

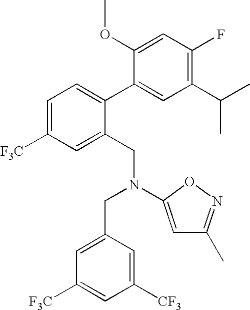

### N-[3,5-bis(trifluoromethyl)benzyl]-N-{[4′-fluoro-5′-isopropyl-2′-methoxy-4-(trifluoromethyl)biphenyl-2-yl]methyl}-3-methylisoxazol-5-amine

To a stirred suspension of potassium tert-butoxide (15 mg, 0.130 mmol) in DMF (1 mL) at 0° C. under an atmosphere of N₂ was added N-[3,5-bis(trifluoromethyl)benzyl]-3-methylisoxazol-5-amine (Intermediate 14; 30 mg, 0.093 mmol) portionwise over 5 min. The resultant solution was stirred at 0° C. for 20 min prior to the addition of a solution of 2′-(bromomethyl)-4-fluoro-5-isopropyl-2-methoxy-4′-(trifluoromethyl)biphenyl (Intermediate 12; 37 mg, 0.093 mmol) in DMF (1 mL). The reaction was allowed to warm to room temperature and stirred for 14 h. The reaction was quenched with H₂O and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-10% EtOAc/hexanes gradient) to afford N-[3,5-bis(trifluoromethyl)benzyl]-N-{[4′-fluoro-5′-isopropyl-2′-methoxy-4-(trifluoromethyl)biphenyl-2-yl]methyl}-3-methylisoxazol-5-amine as a colorless oil. LCMS=649.3 (M+1)⁺. ¹H NMR (CDCl₃, 500 MHz): δ 7.79 (s, 1H), 7.62 (d, J=7.8 Hz, 1H), 7.52 (s, 2H), 7.44 (s, 1H), 7.33 (d, J=7.8 Hz, 1H), 6.94 (d, J=8.5 Hz, 1H), 6.67 (d, J=11.9 Hz, 1H), 4.66 (s, 1H), 4.50-4.30 (m, 4H), 3.70 (s, 3H), 3.22 (m, 1H), 2.18 (s, 3H), 1.27 (d, J=6.9 Hz, 3H), 1.22 (d, J=6.9 Hz, 3H).

### Example 9

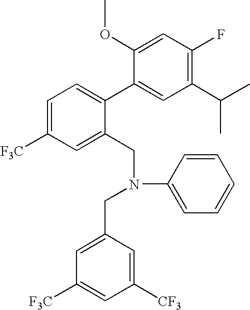

### N-[3,5-bis(trifluoromethyl)benzyl]-N-{[4′-fluoro-5′-isopropyl-2′-methoxy-4-(trifluoromethyl)biphenyl-2-yl]methyl}aniline

To a stirred suspension of potassium tert-butoxide (20 mg, 0.18 mmol) in DMF (2 mL) at 0° C. under an atmosphere of N₂ was added N-[3,5-bis(trifluoromethyl)benzyl]aniline (Intermediate 15; 40 mg, 0.13 mmol) portionwise over 5 min. The resultant solution was stirred at 0° C. for 20 min prior to the addition of a solution of 2′-(bromomethyl)-4-fluoro-5-isopropyl-2-methoxy-4′-(trifluoromethyl)biphenyl (Intermediate 12; 53 mg, 0.13 mmol) in DMF (1 mL). The reaction was allowed to warm to room temperature and stirred for 14 h. The reaction was quenched with H₂O and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-10% EtOAc/hexanes gradient) to afford 31 mg of N-[3,5-bis(trifluoromethyl)benzyl]-N-{[4′-fluoro-5′-isopropyl-2′-methoxy-4-(trifluoromethyl)biphenyl-2-yl]methyl}aniline as a colorless oil. LCMS=644.4 (M+1)⁺. ¹H NMR (CDCl₃, 500 MHz): δ 7.79 (s, 1H), 7.64 (s, 2H), 7.58 (d, J=8.1 Hz, 1H), 7.34 (d, J=8.0 Hz, 1H), 7.22-7.18 (m, 3H), 6.98 (d, J=8.5 Hz, 1H), 6.82 (m, 1H), 6.68-6.62 (m, 2H), 4.66 (d, J=6.8 Hz, 1H), 4.64 (d, J=17.7 Hz, 1H), 4.32-4.26 (d, J=17.4 Hz, 1H), 3.72 (s, 3H), 3.26 (m, 1H), 1.28 (d, J=7.1 Hz, 3H), 1.22 (d, J=7.1 Hz, 3H).

### Example 10

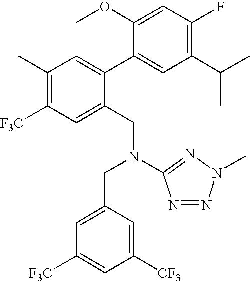

### N-[3,5-bis(trifluoromethyl)benzyl]-N-{[4′-fluoro-5′-isopropyl-2′-methoxy-5-methyl-4-(trifluoromethyl)biphenyl-2-yl]methyl}-2-methyl-2H-tetrazol-5-amine

To a stirred suspension of sodium hydride (60% in oil; 2.4 mg, 0.06 mmol) in THF (1 mL) at 0° C. under an atmosphere of N₂ was added N-[3,5-bis(trifluoromethyl)benzyl]-2-methyl-2H-tetrazol-5-amine (Intermediate 1; 7.8 mg, 0.024 mmol) portionwise. The resultant solution was stirred at 0° C. for 20 min prior to the addition of a solution of 2-(bromomethyl)-4′-fluoro-5′-isopropyl-2′-methoxy-5-methyl-4-(trifluoromethyl)biphenyl (Intermediate 17; 10 mg, 0.024 mmol) in THF (1 mL). The reaction was allowed to warm to room temperature and stirred for 14 h. The reaction was quenched with H₂O (1 mL) and was partitioned between H₂O (15 mL) and EtOAC (25 mL). The aqueous layer was re-extracted with EtOAc (3×25 mL) and the combined extracts were washed with brine (25 mL), dried (MgSO₄), filtered and concentrated in vacuo. The residue was purified by flash silica gel chromatography (0-10% EtOAc/hexanes gradient) to afford N-[3,5-bis(trifluoromethyl)benzyl]-N-{[4′-fluoro-5′-isopropyl-2′-methoxy-5-methyl-4-(trifluoromethyl)biphenyl-2-yl]methyl}-2-methyl-2H-tetrazol-5-amine as a colorless oil. LCMS=664.3 (M+1)⁺. ¹H NMR (CDCl₃, 500 MHz): δ 7.75 (s, 1H), 7.48 (brs, 2H), 7.42 (s, 1H), 7.11 (s, 1H), 6.89 (d, J=8.5 Hz, 1H), 6.58 (d, J=12.1 Hz, 1H), 4.58 (s, 2H), 4.47 (d, J=4.4 Hz, 2H), 4.16 (s, 3H), 3.70 (s, 3H), 3.18-3.13 (m, 1H), 2.47 (s, 3H), 1.22 (d, J=7.0 Hz, 3H), 1.18 (d, J=6.8 Hz, 3H).

### Example 11

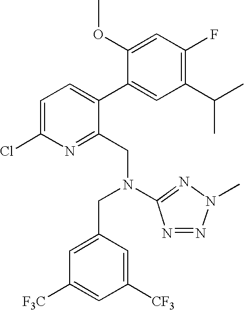

### N-[3,5-bis(trifluoromethyl)benzyl]-N-{[6-chloro-3-(4-fluoro-5-isopropyl-2-methoxyphenyl)pyridine-2-yl]methyl}-2-methyl-2H-tetrazol-5-amine

A mixture of N-[3,5-bis(trifluoromethyl)benzyl]-N-[(3-bromo-6-chloropyridin-2-yl)methyl]-2-methyl-2H-tetrazol-5-amine (Intermediate 16, 307 mg, 0.58 mmol), (4-fluoro-5-isopropyl-2-methoxyphenyl)boronic acid (Intermediate 4, 246 mg, 1.16 mmol) and 1,1-bis(di^{t}butylphosphino)ferrocene palladium dichloride (38 mg, 0.58 mmol) in aqueous potassium carbonate/THF (18 mL, 18 mL) was heated at reflux for 2.5 h under N₂. After cooling to room temperature, the aqueous phase was separated and extracted with EtOAc (3×40 mL). The combined organic layers were dried (Na₂SO₄) and concentrated in vacuo to give the crude product. This was purified by flash chromatography on silica gel (0-15% EtOAc in hexanes gradient) to afford N-[3,5-bis(trifluoromethyl)benzyl]-N-{[6-chloro-3-(4-fluoro-5-isopropyl-2-methoxyphenyl)pyridine-2-yl]methyl}-2-methyl-2H-tetrazol-5-amine LCMS=617.2 (M+1)⁺. ¹H NMR (CDCl₃, 500 MHz): δ 7.87 (d, J=8.0 Hz, 1H), 7.70-7.64 (m, 3H), 7.70 (m, 1H), 6.90 (d, J=12.1 Hz, 1H), 6.68 (d, J=12.1 Hz, 1H), 4.92 (brs, 2H),), 4.08 (s, 3H), 3.84 (s, 2H), 3.68 (s, 3H), 3.22 (m, 1H), 1.26 (d, J=6.8 Hz, 3H), 1.21 (brs, 3H).

### Example 12

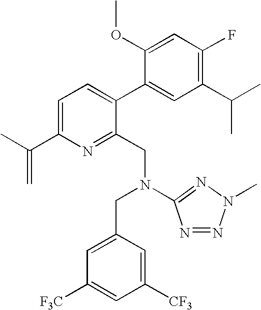

### N-[3,5-bis(trifluoromethyl)benzyl]-N-{[3-(4-fluoro-5-isopropyl-2-methoxyphenyl)-6-isopropenylpyridin-2-yl]methyl}-2-methyl-2H-tetrazol-5-amine

A mixture of N-[3,5-bis(trifluoromethyl)benzyl]-N-{[6-chloro-3-(4-fluoro-5-isopropyl-2-methoxyphenyl)pyridine-2-yl]methyl}-2-methyl-2H-tetrazol-5-amine (Example 11, 285 mg, 0.46 mmol), isopropenyl boronic acid (396 mg, 4.6 mmol) and 1,1-bis(di^{t}butylphosphino)ferrocene palladium dichloride (29 mg, 0.046 mmol) in aqueous potassium carbonate/THF (15 mL, 15 mL) was heated at reflux for 2.5 h under N₂. After cooling to room temperature, the aqueous phase was separated and extracted with EtOAc (3×40 mL). The combined organic layers were dried (Na₂SO₄) and concentrated in vacuo to give the crude product. This was purified by flash chromatography on silica gel (0-15% EtOAc in hexanes gradient) to afford N-[3,5-bis(trifluoromethyl)benzyl]-N-{[3-(4-fluoro-5-isopropyl-2-methoxyphenyl)-6-isopropenylpyridin-2-yl]methyl}-2-methyl-2H-tetrazol-5-amine LCMS=623.2 (M+1)⁺. ¹H NMR (CDCl₃, 500 MHz): δ 7.70 (s, 1H), 7.68 (brs, 2H), 7.53 (d, J=8.0 Hz, 1H), 7.45 (d, J=8.0 Hz, 1H), 6.92 (d, J=8.5 Hz, 1H), 6.62 (d, J=12.1 Hz, 1H), 5.88 (brs, 1H), 5.34 (brs, 1H), 4.85 (brs, 4H), 4.15 (s, 3H), 4.13 (s, 3H), 3.17 (m, 1H), 2.11 (3H, s), 1.29 (brs, 6H).

### Example 13

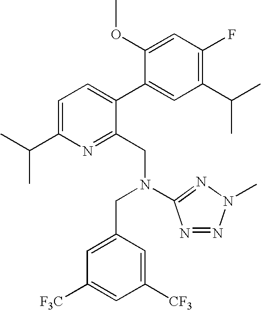

### N-[3,5-bis(trifluoromethyl)benzyl]-N-{[3-(4-fluoro-5-isopropyl-2-methoxyphenyl)-6-isopropylpyridin-2-yl]methyl}-2-methyl-2H-tetrazol-5-amine

A solution of N-[3,5-bis(trifluoromethyl)benzyl]-N-{[3-(4-fluoro-5-isopropyl-2-methoxyphenyl)-6-isopropenylpyridin-2-yl]methyl}-2-methyl-2H-tetrazol-5-amine (Example 12, 34 mg, 0.055 mmol) in EtOH (3 mL) was charged with hydrogen at 1 atm with catalytic amount of Pd/C. The mixture was stirred at room temperature for 1 h. The mixture was filtered through Celite and concentrated. The title compound was obtained after flash chormatography on silica gel using EtOAc:hexane 10:90 as the elute to afford N-[3,5-bis(trifluoromethyl)benzyl]-N-{[3-(4-fluoro-5-isopropyl-2-methoxyphenyl)-6-isopropylpyridin-2-yl]methyl}-2-methyl-2H-tetrazol-5-amine LCMS=625.2 (M+1)⁺. ¹H NMR (CDCl₃, 500 MHz): δ 7.72 (s, 1H), 7.68 (brs, 2H), 7.40 (d, J=7.4 Hz, 1H), 7.11 (d, J=7.7 Hz, 1H), 6.90 (d, J=8.5 Hz, 1H), 6.57 (d, J=12.1 Hz, 1H), 4.74 (brs, 3H), 4.67 (s, 1H), 4.06 (s, 3H), 3.66 (s, 3H), 3.14 (m, 1H), 2.99 (m, 3H), 1.21 (d, J=7.0 Hz, 3H), 1.17 (brs, 3H).

### Example 14

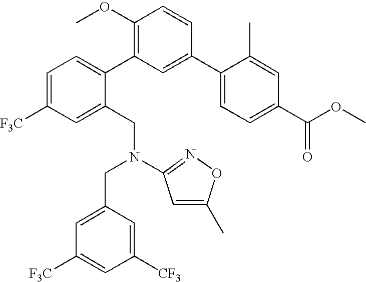

### methyl 2″-{[[3,5-bis(trifluoromethyl)benzyl](5-methylisoxazol-3-yl)amino]methyl}-4′-methoxy-2-methyl-4″-(trifluoromethyl)-1,1′:3′,1″-terphenyl-4-carboxylate

To a solution of N-[3,5-bis(trifluoromethyl)benzyl]-5-methylisoxazol-3-amine (Intermediate 13) (44.8 mg, 0.138 mmol) in DMF (1.8 mL) was added t-BuOK (17.5 mg 0.152 mmol). The reaction was stirred for 15 minutes, then a solution of methyl 2″-(bromomethyl)-4′-methoxy-2-methyl-4″-(trifluoromethyl)-1,1′:3′,1″-terphenyl-4-carboxylate (75 mg, 0.152 mmol) in DMF (1 mL) was added via cannula. The reaction was stirred at room temperature for 1 hour, and then was quenched with saturated NH₄Cl solution (10 mL), diluted with EtOAc (20 mL), washed with brine (10 mL), dried over Na₂SO₄, filtered, and concentrated. The residue was purified by reverse-phase chromatography (C-18, 10 to 95% MeCN/water with 0.1% TFA) to afford methyl 2″-{[[3,5-bis(trifluoromethyl)benzyl}(5-methylisoxazol-3-yl)amino]methyl]-4′-methoxy-2-methyl-4″-(trifluoromethyl)-1,1′:3′,1″-terphenyl-4-carboxylate (as the TFA salt). LCMS=737.0 (M+1)⁴′.

### Example 15

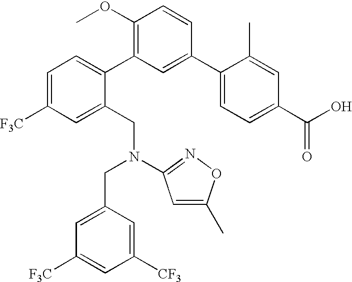

### 2″-{[[3,5-bis(trifluoromethyl)benzyl](5-methylisoxazol-3-yl)amino]methyl}-4′-methoxy-2-methyl-4″-(trifluoromethyl)-1,1′:3′,1″-terphenyl-4-carboxylic acid

To a solution of methyl 2″-{[[3,5-bis(trifluoromethyl)benzyl](5-methylisoxazol-3-yl)amino]methyl}-4′-methoxy-2-methyl-4″-(trifluoromethyl)-1,1′:3′,1″-terphenyl-4-carboxylate from Example 14 (52 mg, 0.0707 mmol) in MeOH (2 mL) was added 4 M KOH solution (1 mL). The reaction was stirred at room temperature for 10 hours, then was quenched with 1 N HCl (5 mL), and then was diluted with EtOAc (15 mL). The aqueous layer was extracted with EtOAc (10 mL), and the combined organic extracts were washed with brine (10 mL), dried over Na₂SO₄, filtered, and concentrated. The residue was purified by reverse-phase chromatography (C-18, 10 to 95% MeCN/water with 0.1% TFA) to afford 2″-{[[3,5-bis(trifluoromethyl)benzyl](5-methylisoxazol-3-yl)amino]methyl}-4′-methoxy-2-methyl-4″-(trifluoromethyl)-1,1′:3′,1″-terphenyl-4-carboxylic acid (as the TFA salt). LCMS=723.1 (M+1)⁺. ¹H NMR (CDCl₃, 500 MHz) δ 8.00 (s, 1H), 7.95 (d, J=8.0 Hz, 1H), 7.72 (s, 1H), 7.59 (d, J=8.2 Hz, 1H), 7.56 (s, 2H), 7.48 (s, 1H), 7.34-7.37 (m, 2H), 7.28 (d, J=8.0 Hz, 1H), 7.06 (d, J=2.1 Hz, 1H), 7.01 (d, J=8.5 Hz, 1H), 6.73 (bs), 5.43 (s, 1H), 4.48-4.52 (m, 3H), 4.34 (d, J=16.5 Hz, 1H), 3.76 (s, 3H), 2.32 (s, 3H), 2.29 (s, 3H).

### Example 16

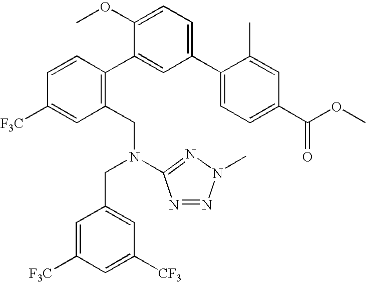

### methyl 2″-{[[3,5-bis(trifluoromethyl)benzyl](2-methyl-2H-tetrazol-5-yl)amino]methyl}-4′-methoxy-2-methyl-4″-(trifluoromethyl)-1,1′:3′,1″-terphenyl-4-carboxylate

N-[3,5-bis(trifluoromethyl)benzyl]-N-[2-iodo-5-(trifluoromethyl)benzyl]-2-methyl-2H-tetrazol-5-amine (Intermediate 10, 83 mg, 0.136 mmol, in 830 μL 1,4-dioxane), methyl 4′-methoxy-2-methyl-3′-(4,4,5,5-tetramethyl-1,3,2-dioxaborolan-2-yl)biphenyl-4-carboxylate (104 mg, 0.272 mmol), 1,1′-bis(diphenylphosphino)ferrocene-palladium dichloride dichloromethane adduct (22 mg, 0.027 mmol), aqueous potassium carbonate (272 μM, 1M, 0.272 mmol) and acetone (1 mL) were combined and stirred in an 82° C. oil bath for 30 minutes. The crude product was cooled (ice bath) and dried (Na₂SO₄). The resulting dark mixture was purified by prep-TLC (SiO₂) developed by elution with 25% ethyl acetate in hexanes (v/v) to afford a yellow glass. This yellow glass was further purified by prep-TLC (SiO₂, 80% DCM (dichloromethane) in hexanes, v/v) to afford a clear glass. This glass was again purified by preparative HPLC (Kromasil 100-5C18, 100×21.1 mm), eluting with MeCN (0.1% TFA, v/v)/Water (0.1% TFA, v/v) (10% to 100% organic in 10 min, hold 100% for 2 min, 20 mL/min), to afford the titled compound as a colorless glass.

### Example 17

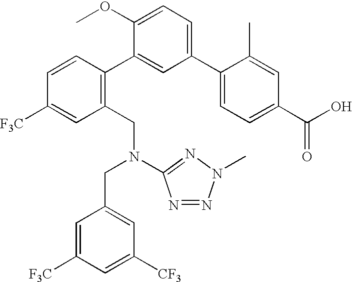

### 2″-{[[3,5-bis(trifluoromethyl)benzyl] (2-methyl-2H-tetrazol-5-yl)amino]methyl}-4′-methoxy-2-methyl-4″-(trifluoromethyl)-1,1′:3′,1″-terphenyl-4-carboxylic acid

Methyl 2″-{[[3,5-bis(trifluoromethyl)benzyl](2-methyl-2H-tetrazol-5-yl)amino]methyl}-4′-methoxy-2-methyl-4″-(trifluoromethyl)-1,1′:3′,1″-terphenyl-4-carboxylate from Example 16 (31.0 mg, 0.042 mmol), lithium hydroxide monohydrate (9 mg, 0.21 mmol), water (0.4 mL) and 1,4-dioxane (1 mL) were stirred at room temperature for 5 hours. The crude mixture was acidified with HCl (aq, 1N, 1 mL). The resulting mixture was worked up with brine and extracted with ethyl acetate. The combined extracts were back-washed with water. The resulting organic layer was dried over Na₂SO₄, filtered and the solvent evaporated in vacuo to afford a clear oil. The resulting oil was purified using a reverse-phase prep-HPLC (Kromasil 100-5C18, 100×21.1 mm) eluted with a MeCN (0.1% TFA, v/v)/H₂O (0.1% TFA, v/v) gradient mixture (10% to 100% organic in 10 min, hold 100% for 2 min, 20 mL/min) to afford the titled compound as a glass. LCMS calc.=723.19; found=724.32 (M+1)⁺. ¹H NMR (CDCl₃, 500 MHz) δ 8.00 (s, 1H), 7.95 (d, J=6.5 Hz, 1H), 7.70 (s, 1H), 7.58 (d, J=7.0 Hz, 1H), 7.52 (s, 1H), 7.49 (s, 1H), 7.37 (d, J=6.5 Hz, 1H), 7.31 (dd, J=2.0, 7.0 Hz, 1H), 7.29 (d, J=7.0 Hz, 1H), 7.07 (d, J=2.0 Hz, 1H), 6.97 (d, J=7.0 Hz, 1H), 4.69 (s, 2H), 4.54 (s, 2H), 4.12 (s, 3H), 3.79 (s, 3H), 2.32 (s, 3H).

1. A compound having Formula I, or a pharmaceutically acceptable salt thereof, wherein 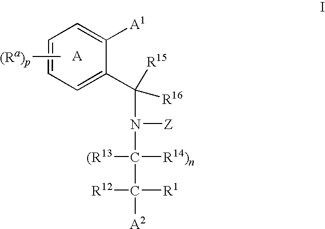
The phenyl ring A of Formula I optionally has —N═ in place of —(CH)═ at one of the 4 positions that is not attached to A1 or —CR15R16- in Formula I;
A1 is selected from the group consisting of:
(a) an aromatic ring selected from phenyl and naphthyl;
(b) a phenyl ring fused to a 5-7 membered non-aromatic cycloalkyl ring, which optionally comprises 1-2 double bonds;
(c) a 5-6-membered heterocyclic ring having 1-4 heteroatoms independently selected from N, S, and O, and optionally also comprising 1-3 double bonds and optionally a carbonyl group or —N(O)— group, wherein the point of attachment of A1 to the phenyl ring A is a carbon atom of A1; and
(d) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A1 to the phenyl ring A is a carbon atom of A1;
wherein A1 is optionally substituted with 1-5 substituent groups independently selected from Rb;
A2 is selected from the group consisting of:
(a) an aromatic ring selected from phenyl and naphthyl;
(b) a phenyl ring fused to a 5-7 membered non-aromatic cycloalkyl ring, which optionally comprises 1-2 double bonds;
(c) a 5-6-membered heterocyclic ring having 1-4 heteroatoms independently selected from N, S, and O, and optionally also comprising 1-3 double bonds and optionally a carbonyl group or —N(O)— group, wherein the point of attachment of A2 is a carbon atom of A2;
(d) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A2 is a carbon atom of A2; and
(e) a —C3-C8 cycloalkyl ring optionally having 1-3 double bonds;
wherein A2 is optionally substituted with 1-5 substituent groups independently selected from Rc;
Each Ra and Rc is independently selected from the group consisting of —C1-C6 alkyl, —C2-C6 alkenyl, —C2-C6 alkynyl, —C3-C8 cycloalkyl optionally having 1-3 double bonds, —OC1-C6alkyl, —OC2-C6 alkenyl, —OC2-C6 alkynyl, —OC3-C8 cycloalkyl optionally having 1-3 double bonds, —C(═O)C1-C6alkyl, —C(═O)C3-C8 cycloalkyl, —C(═O)H, —CO2H, —CO2C1-C6alkyl, —C(═O)SC1-C6alkyl, —NR10R11, —C(═O)NR10R11, —NR10C(═O)OC1-C6 alkyl, —NR10C(═O)NR10R11, —S(O)xC1-C6 alkyl, —S(O)yNR10R11, —NR10S(O)yNR10R11, halogen, —CN, —NO2, and a 5-6-membered heterocyclic ring having 1-4 heteroatoms independently selected from N, S, and O, said heterocyclic ring optionally also comprising a carbonyl group and optionally 1-3 double bonds,
wherein when Ra and Rc are selected from the group consisting of a heterocyclic ring, —C3-C8 cycloalkyl, —OC3-C8 cycloalkyl, and —C(═O)C3-C8 cycloalkyl, the heterocyclic ring and —C3-C8 cycloalkyl groups of Ra and Rc are optionally substituted with 1-5 substituent groups independently selected from halogen, —C1-C3 alkyl, and —OC1-C3 alkyl, wherein —C1-C3 alkyl and —OC1-C3 alkyl are optionally substituted with 1-7 halogens,
and when Ra and Rc are selected from the group consisting of —C1-C6 alkyl, —C2-C6 alkenyl, —C2-C6 alkynyl, —OC1-C6alkyl, —OC2-C6 alkenyl, —OC2-C6 alkynyl, —C(═O)C1-C6alkyl, —CO2C1-C6alkyl, —C(═O)SC1-C6alkyl, —NR10C(═O)OC1-C6 alkyl, and —S(O)xC1-C6 alkyl, the alkyl, alkenyl, and alkynyl groups of Ra and Rc are optionally substituted with 1-13 halogens and are optionally substituted with 1-3 substituent groups independently selected from (a) —OH, (b) —CN, (c) —NR10R11, (d) —C3-C8 cycloalkyl optionally having 1-3 double bonds and optionally substituted with 1-15 halogens, (e) —OC1-C4alkyl optionally substituted with 1-9 halogens and optionally substituted with 1-2 substituent groups independently selected from —OC1-C2 alkyl, (f) —OC3-C8 cycloalkyl optionally having 1-3 double bonds and optionally substituted with 1-15 halogens, (g) —CO2H, (h) —C(═O)CH3, and (i) —CO2C1-C4alkyl which is optionally substituted with 1-9 halogens;
wherein 2 groups Ra that are on adjacent carbon atoms of the phenyl or optional pyridyl ring A of Formula I may optionally be joined to form a bridging group selected from —CH2CH2CH2-, —CH2CH2CH2CH2-, and —CH═CH—CH═CH—, thereby yielding a cyclopentyl, cyclohexyl, or phenyl ring fused to the phenyl ring or optional pyridyl ring A of Formula I, wherein said cyclopentyl, cyclohexyl, or phenyl ring that is fused to the phenyl or optional pyridyl ring A of Formula I is optionally substituted with 1-2 groups Ra, with the proviso that two adjacent groups Ra cannot be joined to form an additional fused ring;
Each Rb is independently selected from the group consisting of —C1-C6 alkyl, —C2-C6 alkenyl, —C2-C6 alkynyl, —C3-C8 cycloalkyl optionally having 1-3 double bonds, —OC1-C6alkyl, —OC2-C6 alkenyl, —OC2-C6 alkynyl, —OC3-C8 cycloalkyl optionally having 1-3 double bonds, —C(═O)C1-C6alkyl, —C(═O)C3-C8 cycloalkyl, —C(═O)H, —CO2H, —CO2C1-C6alkyl, —C(═O)SC1-C6alkyl, —NR10R11, —C(═O)NR10R11, —NR10C(═O)OC1-C6 alkyl, —NR10C(═O)NR10R11, —S(O)xC1-C6 alkyl, —S(O)yNR10R11, —NR10S(O)yNR10R11, halogen, —CN, —NO2, phenyl, and a 5-6-membered heterocyclic ring having 1-4 heteroatoms independently selected from N, S, and O, said heterocyclic ring optionally also comprising a carbonyl group and optionally 1-3 double bonds,
wherein when Rb is selected from the group consisting of a heterocyclic ring, —C3-C8 cycloalkyl, —OC3-C8 cycloalkyl, and —C(═O)C3-C8 cycloalkyl, the heterocyclic ring and —C3-C8 cycloalkyl groups of Rb are optionally substituted with 1-5 substituent groups independently selected from halogen, —C1-C3 alkyl, —C2-C3 alkenyl, —NR10R11, —OC1-C3 alkyl, —CO2H, —CN, and —CO2C1-C3alkyl, wherein —C1-C3 alkyl and —C2-C3 alkenyl in all uses are optionally substituted with 1-7 halogens and optionally one group —OH, and when Rb is selected from the group consisting of —C1-C6 alkyl, —C2-C6 alkenyl, —C2-C6 alkynyl, —OC1-C6alkyl, —OC2-C6 alkenyl, —OC2-C6 alkynyl, —C(═O)C1-C6alkyl, —CO2C1-C6alkyl, —C(═O)SC1-C6alkyl, —NR10C(═O)OC1-C6 alkyl, and —S(O)xC1-C6 alkyl, the alkyl, alkenyl, and alkynyl groups of Rb are optionally substituted with 1-13 halogens and are optionally substituted with 1-3 substituent groups independently selected from (a) —OH, (b) —CN, (c) —NR10R11, (d) —C3-C8 cycloalkyl optionally having 1-3 double bonds and optionally substituted with 1-15 halogens, (e) —OC1-C4alkyl optionally substituted with 1-9 halogens and optionally substituted with 1-2 substituent groups independently selected from —OC1-C2 alkyl, (f) —OC3-C8 cycloalkyl optionally having 1-3 double bonds and optionally substituted with 1-15 halogens, (g) —CO2H, (h) —C(═O)CH3, and (i) —CO2C1-C4alkyl which is optionally substituted with 1-9 halogens,
and when Rb is phenyl, said phenyl is optionally substituted with 1-5 halogens and is optionally substituted with 1-3 substituents independently selected from C1-C4 alkyl, —C2-C4 alkenyl, —C2-C4 alkynyl, —C3-C6 cycloalkyl, —OC1-C4alkyl, —OC2-C4 alkenyl, —OC2-C4 alkynyl, —OC3-C6 cycloalkyl, —C(═O)C1-C4alkyl, —C(═O)H, —CO2H, —CO2C1-C4alkyl, —NR10R11, —C(═O)NR10R11, —NR10C(═O)OC1-C4 alkyl, —NR10C(═O)NR10R11, —S(O)xC1-C4 alkyl, —S(O)yNR10R11, —NR10S(O)yNR10R11, —CN, —NO2, and a 5-6-membered heterocyclic ring having 1-4 heteroatoms independently selected from N, S, and O, said heterocyclic ring optionally also comprising a carbonyl group and optionally 1-3 double bonds and optionally 1-3 substituents independently selected from halogen, —CH3, —OCH3, —CF3, and —OCF3; wherein when the substituents on phenyl when Rb is phenyl are selected from —C1-C4 alkyl, —C2-C4 alkenyl, —C2-C4 alkynyl, —C3-C6 cycloalkyl, —OC1-C4alkyl, —OC2-C4 alkenyl, —OC2-C4 alkynyl, —OC3-C6 cycloalkyl, —C(═O)C1-C4alkyl, —CO2C1-C4alkyl, —NR10C(═O)OC1-C4 alkyl, and —S(O)xC1-C4 alkyl, then the alkyl, alkenyl, alkynyl, and cycloalkyl groups of said substituent groups optionally comprise 1-5 halogen substituents and optionally one substituent selected from —OH, —NR10R11, —OCH3 which is optionally substituted with 1-3 F, and phenyl which is optionally substituted with 1-3 substituents independently selected from halogen, —CH3, —OCH3, —CF3, and —OCF3;
n is an integer selected from 0 and 1;
p is an integer from 0-4;
x is an integer selected from 0, 1, and 2;
y is an integer selected from 1 and 2;
Z is phenyl or a 5-6-membered heterocyclic ring having 1-4 heteroatoms independently selected from N, S, and O, said heterocyclic ring optionally comprising a carbonyl group and 1-3 double bonds, said heterocyclic ring being connected by a carbon atom to the N to which said heterocyclic ring is attached, wherein said phenyl or 5-6-membered heterocyclic ring optionally comprises 1-3 substituents independently selected from halogen, C1-C4 alkyl, —C2-C4 alkenyl, —C2-C4 alkynyl, —OC1-C4alkyl, —OC2-C4 alkenyl, —OC2-C4 alkynyl, —C(═O)C1-C4alkyl, CO2C1-C4alkyl, —NR10R11, —C(═O)NR10R11, —NR10C(═O)OC1-C4 alkyl, —NR10C(═O)NR10R11, —S(O)xC1-C4 alkyl, —S(O)yNR10R11, —NR10S(O)yNR10R11, —OH, —CN, and —NO2, wherein the alkyl, alkenyl, and alkynyl groups of said substituents are optionally substituted with 1-5 halogens and optionally one substituent selected from —OH, —CO2H, and —CO2C1-C4alkyl;
R1, R12, R13, R14, R15, and R16 are each independently selected from the group consisting of H, —OH, halogen, —C1-C4 alkyl, —C3-C6 cycloalkyl, —OC1-C4 alkyl, and —NR10R11, wherein —C1-C4 alkyl, —C3-C6 cycloalkyl, and —OC1-C4 alkyl are each optionally substituted with 1-9 halogens and are each optionally substituted with 1-2 groups independently selected from —OH, —C(═O)CH3, —OC(═O)CH3, —OC1-C2 alkyl, and —OC1-C2 alkyleneOC1-C2alkyl, wherein R1 and R12 together may optionally form an oxo group; and
R10 and R11 are each independently selected from H, —C1-C5 alkyl, —C(═O)C1-C5 alkyl and —S(O)yC1-C5 alkyl, wherein —C1-C5 alkyl in all instances is optionally substituted with 1-11 halogens.

2. The compound of claim 1, or a pharmaceutically acceptable salt thereof, wherein
The phenyl ring A of Formula I optionally has —N═ in place of —(CH)═ at one of the 4 positions of the phenyl ring that is not attached to A1 or —CR15R16- in Formula I;
A1 is selected from the group consisting of:
(a) an aromatic ring selected from phenyl and naphthyl;
(b) a 5-6-membered heterocyclic ring having 1-4 heteroatoms independently selected from N, S, and O, and optionally also comprising 1-3 double bonds and optionally a carbonyl group or —N(O)— group, wherein the point of attachment of A1 to the phenyl ring A is a carbon atom of A1; and
(c) a benzoheterocyclic ring comprising a phenyl ring fused to a 5-6-membered heterocyclic ring having 1-3 heteroatoms independently selected from O, N, and S, and optionally 1-2 double bonds, wherein the point of attachment of A1 to the phenyl ring A is a carbon atom of A1;
wherein A1 is optionally substituted with 1-4 substituent groups independently selected from —C1-C5 alkyl, —OC1-C3alkyl, —CO2C1-C3alkyl, —CO2H, halogen, —NR10R11, —C(═O)C1-C3alkyl, —C(═O)H, —C(═O)NR10R11, —S(O)xC1-C3 alkyl, —C2-C3 alkenyl, —CN, —NO2, —C3-C6 cycloalkyl, phenyl, and a 5-6-membered heterocyclic ring having 1-3 heteroatoms independently selected from N, S, and O, and optionally also comprising 1-3 double bonds, wherein —C1-C3 alkyl, —C1-C5 alkyl, and —C2-C3 alkenyl in all occurrences are optionally substituted with 1-6 substituents independently selected from 1-5 halogens and one —OH or —CO2H group; and —C3-C6 cycloalkyl, phenyl, and the 5-6-membered heterocyclic ring are optionally substituted with 1-3 substituents independently selected from halogen, —C1-C3 alkyl, —C2-C3 alkenyl, —OC1-C3 alkyl, —NR10R11, —CO2H, —CO2C1-C3 alkyl, and —CN, wherein —C1-C3 alkyl and —C2-C3 alkenyl in all uses are optionally substituted with 1-3 halogens and optionally one —OH group;
A2 is selected from the group consisting of phenyl, naphthyl, —C3-C6 cycloalkyl, and a heterocyclic 5-6 membered ring having 1-3 heteroatoms independently selected from O, N, and S, and optionally also comprising 1-3 double bonds and optionally a carbonyl group or —N(O)—group, wherein A2 is optionally substituted with 1-2 substituent groups independently selected from —C1-C4 alkyl, —OC1-C3 alkyl, —C(═O)C1-C3alkyl, —C(═O)H, —NO2, —CN, —S(O)xC1-C3 alkyl, —NR10R11, —NR10C(═O)R11, —C2-C3 alkenyl, —C(═O)NR10R11, halogen, —C3-C6 cycloalkyl, and a 5-6-membered heterocyclic ring having 1-3 heteroatoms independently selected from N, S, and O, and optionally also comprising 1-3 double bonds, wherein C1-C3 alkyl, C1-C4 alkyl, and C2-C3alkenyl in all instances are optionally substituted with 1-3 halogens, and —C3-C6 cycloalkyl and the 5-6-membered heterocyclic ring are optionally substituted with 1-3 substituents independently selected from halogen and —C1-C3 alkyl;
Each Ra is independently selected from the group consisting of H, halogen, —NR10R11, —C1-C3 alkyl, —OC1-C3 alkyl, —C2-C3 alkenyl, —C3-C6 cycloalkyl optionally having a double bond, —OC3-C6 cycloalkyl optionally having a double bond, —C(═O)C1-C3alkyl, —C(═O)C3-C6 cycloalkyl, —C(═O)H, —CO2H, —CO2C1-C3alkyl, —C(═O)NR10R11, —CN, —NO2, and a 5-6-membered heterocyclic ring having 1-4 heteroatoms independently selected from N, S, and O, and optionally 1-3 double bonds, wherein C1-C3 alkyl and —C2-C3 alkenyl in all instances are optionally substituted with 1-5 halogens, and —C3-C6 cycloalkyl and the 5-6-membered heterocyclic ring are in all occurrences optionally substituted with 1-3 substituents independently selected from halogen, —C1-C3 alkyl, —OC1-C3 alkyl, —CF3, and —OCF3;
wherein 2 groups Ra that are on adjacent carbon atoms of the phenyl or optional pyridyl ring A of Formula I may optionally be joined to form a bridging group selected from —CH2CH2CH2-, —CH2CH2CH2CH2-, and —CH═CH—CH═CH—, thereby yielding a cyclopentyl, cyclohexyl, or phenyl ring fused to the phenyl ring or optional pyridyl ring A of Formula I, wherein said cyclopentyl, cyclohexyl, or phenyl ring that is fused to the phenyl or optional pyridyl ring A of Formula I is optionally substituted with 1-2 groups Ra, with the proviso that two adjacent groups Ra cannot be joined to form an additional fused ring;
n is an integer selected from 0 and 1;
p is an integer selected from 0-4;
x is an integer selected from 0, 1, and 2;
R1 is selected from the group consisting of H, F, OH, C1-C3 alkyl, and —OC1-C3 alkyl, wherein C1-C3 alkyl and —OC1-C3 alkyl are each optionally substituted with 1-3 halogens and are optionally substituted with one —OC1-C2alkyl;
R10 and R11 are each independently selected from H and —C1-C3 alkyl;
R12, R13, R14, R15, and R16 are each H; and
Z is selected from phenyl and a 5-6-membered heterocyclic ring having 1-4 heteroatoms independently selected from N, S, and O, said heterocyclic ring optionally also comprising 1-3 double bonds, said heterocyclic ring being connected by a carbon atom to the N to which said heterocyclic ring is attached, wherein said phenyl or 5-6-membered heterocyclic ring optionally comprises 1-3 substituents independently selected from halogen, C1-C4 alkyl, —C2-C4 alkenyl, —C2-C4 alkynyl, —OC1-C4alkyl, —OC2-C4 alkenyl, —OC2-C4 alkynyl, —C(═O)C1-C4alkyl, —CO2C1-C4alkyl, —NR10R11, —OH, —CN, and —NO2, wherein the alkyl, alkenyl, and alkynyl groups of said substituents are optionally substituted with 1-5 halogens and optionally one substituent selected from —OH, —CO2H, and —CO2C1-C4alkyl.

3. The compound of claim 1 having Formula Ia, or a pharmaceutically acceptable salt thereof: 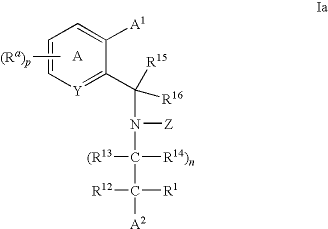
wherein Y is selected from the group consisting of —N═ and —CH═.

4. The compound of claim 2 having Formula Ib, or a pharmaceutically acceptable salt thereof, 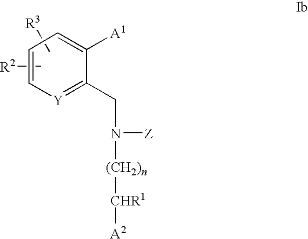
wherein Y is selected from the group consisting of —N═ and —CH═; and
R2, R3, and Ra are each independently selected from the group consisting of H, halogen, —NR10R11, —C1-C3 alkyl, —OC1-C3 alkyl, —C2-C3 alkenyl, —C3-C6 cycloalkyl optionally having a double bond, —OC3-C6 cycloalkyl optionally having a double bond, —C(═O)C1-C3alkyl, —C(═O)C3-C6 cycloalkyl, —C(═O)H, —CO2H, —CO2C1-C3alkyl, —C(═O)NR10R11, —CN, —NO2, and a 5-6-membered heterocyclic ring having 1-4 heteroatoms independently selected from N, S, and O, and optionally 1-3 double bonds, wherein C1-C3 alkyl and —C2-C3 alkenyl in all instances are optionally substituted with 1-5 halogens, and —C3-C6 cycloalkyl and the 5-6-membered heterocyclic ring are in all occurrences optionally substituted with 1-3 substituents independently selected from halogen, —C1-C3 alkyl, —OC1-C3 alkyl, —CF3, and —OCF3;
wherein when R2 and R3 are on adjacent carbon atoms of the phenyl or optional pyridyl ring of Formula Ib, then R2 and R3 may optionally be joined to form a bridging group selected from —CH2CH2CH2-, —CH2CH2CH2CH2-, and —CH═CH—CH═CH—, thereby yielding a cyclopentyl, cyclohexyl, or phenyl ring fused to the phenyl ring or optional pyridyl ring of Formula Ib, wherein said cyclopentyl, cyclohexyl, or phenyl ring that is fused to the phenyl or optional pyridyl ring of Formula Ib is optionally substituted with 1-2 groups Ra, and the remaining 1 or 2-CH═ positions of the phenyl or optional pyridyl ring of Figure Ib are optionally substituted with Ra, with the proviso that two adjacent groups Ra cannot be joined to form an additional fused ring.

5. The compound of claim 4, or a pharmaceutically acceptable salt thereof, wherein R2, R3, and Ra are each independently selected from the group consisting of H, halogen, —NR10R11, —C1-C3 alkyl, —C2-C3 alkenyl, —OC1-C3 alkyl, —CN, —NO2, and pyridyl, wherein C1-C3 alkyl and —C2-C3 alkenyl in all instances are optionally substituted with 1-3 halogens, and pyridyl is optionally substituted with 1-3 substituents independently selected from the group consisting of halogen, —CH3, —CF3, —OCH3, and —OCF3.

6. The compound of claim 4 having the Formula Ic, or a pharmaceutically acceptable salt thereof: 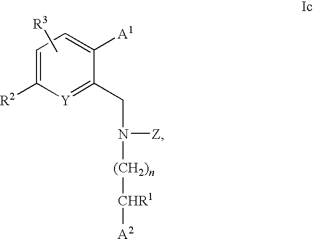
wherein Y is selected from the group consisting of —N═ and —CH═; and
R2 and R3 cannot be joined to form an additional fused ring.

7. The compound of claim 6, or a pharmaceutically acceptable salt thereof:
wherein Y is selected from the group consisting of —N═ and —CH═;
R2 is selected from the group consisting of H, halogen, cyclopropyl, —NR10R11, —C1-C3 alkyl, —C2-C3 alkenyl, —OC1-C3 alkyl, —CN, —NO2, and pyridyl, wherein cyclopropyl, C1-C3 alkyl and C2-C3 alkenyl in all instances are optionally substituted with 1-3 halogens, and pyridyl is optionally substituted with 1-3 substituents independently selected from the group consisting of halogen, —CH3, —CF3, —OCH3, and —OCF3;
R3 is selected from the group consisting of H, halogen, —CH3, —CF3, —OCH3, and —OCF3; and R2 and R3 cannot be joined to form a fused ring.

8. (canceled)

9. The compound of claim 6, or a pharmaceutically acceptable salt thereof, wherein A1 is selected from the group consisting of phenyl, thienyl, furyl, pyridinyl, quinolyl, isoquinolyl, benzofuranyl, dihydrobenzofuranyl, indolyl, dihydroindolyl, oxazolyl, and isoxazolyl; and
A2 is selected from phenyl, pyridinyl, thienyl, 1-oxidopyridinyl, and cyclohexyl;
wherein A1 and A2 are optionally substituted as in claim 6.

10. The compound of claim 7, or a pharmaceutically acceptable salt thereof, wherein A1 and A2 are phenyl, wherein A1 and A2 are substituted as in claim 7, and R1 is H or CH3.

11. The compound of claim 10, or a pharmaceutically acceptable salt thereof, wherein R1 is H and n is 0.

12. The compound of claim 1, or a pharmaceutically acceptable salt thereof, wherein Z is selected from the group consisting of phenyl, tetrazolyl, oxadiazolyl, thiadiazolyl, 1,2,3-triazolyl, 1,2,4-triazolyl, oxazolyl, isoxazolyl, thiazolyl, isothiazolyl, pyrazolyl, imidazolyl, thienyl, furyl, pyridinyl, pyrimidinyl, pyrazinyl, and dioxinyl, wherein Z is optionally substituted with 1-3 substituents independently selected from halogen, C1-C4 alkyl, —C2-C4 alkenyl, —C2-C4 alkynyl, —OC1-C4alkyl, —OC2-C4 alkenyl, —OC2-C4 alkynyl, —C(═O)C1-C4alkyl, —CO2C1-C4alkyl, —NR10R11, —OH, —CN, and —NO2, wherein the alkyl, alkenyl, and alkynyl groups of said substituents are optionally substituted with 1-5 halogens and optionally one substituent selected from —OH, —CO2H, and —CO2C1-C4alkyl.

13-14. (canceled)

15. The compound of claim 1 having formula II, or a pharmaceutically acceptable salt thereof, wherein 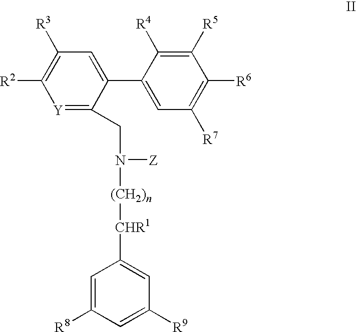
Y is selected from the group consisting of —N═ and —CH═;
R1 is selected from the group consisting of H and CH3;
R2 is selected from the group consisting of H, halogen, —NR10R11, —OC1-C3 alkyl, C1-C3 alkyl, C2-C3 alkenyl, —CN, —NO2, and pyridyl, wherein C1-C3 alkyl and C2-C3 alkenyl in all occurrences are optionally substituted with 1-3 halogens;
R3 is selected from the group consisting of H and —C1-C3 alkyl, which is optionally substituted with 1-3 F;
R2 and R3 do not have the option of joining to form a bridging group selected from —CH2CH2CH2-, —CH2CH2CH2CH2-, and —CH═CH—CH═CH—;
R4 is selected from the group consisting of H, halogen, —C1-C3 alkyl, —OC1-C3 alkyl, —SC1-C2 alkyl and —CN, wherein —C1-C3 alkyl, —SC1-C3 alkyl, and —OC1-C3 alkyl are optionally substituted with 1-3 F;
R5 and R6 are each independently selected from the group consisting of H, halogen, —CH3 and —OCH3, wherein —CH3 and —OCH3 are optionally substituted with 1-3 F;
R7 is selected from the group consisting of H, —C1-C5alkyl, —OC1-C3 alkyl, —C2-C3 alkenyl, halogen, —CN, —CO2H, —CO2C1-C3 alkyl, —SC1-C3 alkyl, —C(═O)NR10R11, —C(═O)H, —C(═O)C1-C3 alkyl, C3-C6 cycloalkyl, phenyl, and 5-(1,2,4-oxadiazolyl),
wherein —C1-C3 alkyl and —C1-C5 alkyl in all occurrences are optionally substituted with 1-6 substituent groups independently selected from 1-5 halogens and one —OH,
—C2-C3 alkenyl is optionally substituted with 1-3 halogens,
1,2,4-oxadiazolyl and C3-C6 cycloalkyl are optionally substituted with 1-2 substituent groups independently selected from halogen, C1-C3 alkyl, and CF3, and
phenyl is optionally substituted with 1-3 substituents independently selected from halogen, —C1-C3 alkyl, —C2-C3 alkenyl, —OC1-C3 alkyl, —NR10R11, —CO2H, —CO2C1-C3 alkyl, and —CN, wherein —C1-C3 alkyl and —C2-C3 alkenyl in all uses are optionally substituted with 1-3 halogens;
R8 and R9 are each independently selected from the group consisting of H, —C1-C3 alkyl, halogen, —S(O)xC1-C3 alkyl, —NR10R11, —OC1-C3alkyl, C2-C3 alkenyl, —NO2, —CN, —C(O)NR10R11, —C(═O)H, —NHC(═O)C1-C3 alkyl, —NHS(O)2C1-C3 alkyl, CO2H, CO2C1-3alkyl, C3-C6 cycloalkyl, and pyridyl, wherein C1-C3 alkyl in all occurrences is optionally substituted with 1-3 halogens, C2-C3 alkenyl is optionally substituted with 1-3 halogens, and C3-C6 cycloalkyl and pyridyl are optionally substituted with 1-2 substituent groups independently selected from halogen and C1-C3 alkyl;
R10 and R11 are each H or C1-C3 alkyl;
Z is selected from the group consisting of phenyl, tetrazolyl, 1,2,3-triazolyl, 1,2,4-triazolyl, thiadiazolyl, oxadiazolyl, oxazolyl, isoxazolyl, pyrazolyl, imidazolyl, thiazolyl, isothiazolyl, pyridyl, and pyrimidinyl, which are optionally substituted with 1-3 substituents independently selected from —CH3 and —CF3;
n is an integer selected from 0 and 1; and
x is an integer selected from 0, 1 and 2.

16. The compound of claim 15, or a pharmaceutically acceptable salt thereof, wherein
R2 is selected from the group consisting of —OCF3, —OCH3, —NO2, —CN, halogen, C1-C3alkyl, C2-C3alkenyl, —NH2 and 3-pyridyl, wherein —C1-C3 alkyl and —C2-C3 alkenyl in all uses are optionally substituted with 1-3 F;
R3 is H or CH3;
R4 is selected from the group consisting of H, halogen, C1-C3alkyl, C2-C3alkenyl, —OCH3, —OCF3, —OC2H5, —SCH3, and —CN;
R5 is selected from the group consisting of H and F;
R6 is selected from the group consisting of H, F, —CH3, and —OCH3;
R7 is selected from the group consisting of H, C1-C4alkyl, —C(═O)H, —C(═O)CH3, —CH═CH2, —CN, Cl, F, —CO2H, —CO2C1-C3alkyl, —OCH3, —SCH3, —C(═O)NR10R11, 3-methyl-5-(1,2,4-oxadiazolyl), and phenyl, wherein C1-C4alkyl and C1-C3alkyl are optionally substituted with 1-6 substituents which are independently selected from 1-5 F and one —OH, and wherein phenyl is optionally substituted with 1-3 substituents independently selected from the group consisting of halogen, —C1-C3 alkyl, —C2-C3 alkenyl, —OC1-C3 alkyl, —NR10R11, —CO2H, —CO2C1-C3 alkyl, and —CN, wherein —C1-C3 alkyl and —C2-C3 alkenyl in all uses are optionally substituted with 1-3 halogens;
R8 and R9 are each independently selected from the group consisting of H, C1-C2alkyl, which is optionally substituted with 1-3 F; halogen; —CN; —NO2; —S(O)xCH3, which is optionally substituted with 1-3F; —OCH3, which is optionally substituted with 1-3 F; —CH═CH2; —C(═O)H; —C(═O)NR10R11; —CO2H; —NR10R11; —CO2C1-C3alkyl; —NHC(═O)CH3; —NHS(O)2CH3; and 4-pyridyl; and
R10 and R11 are each independently selected from H and CH3.

17. The compound of claim 16, or a pharmaceutically acceptable salt thereof, wherein R1 is H and n is 0, or a pharmaceutically acceptable salt thereof.

18-24. (canceled)

25. The compound of claim 1, or a pharmaceutically acceptable salt thereof, wherein Z is selected from the group consisting of tetrazolyl, isoxazolyl, triazolyl, pyrazolyl, oxadiazolyl, and thiadiazolyl, which are optionally substituted with 1-3 substituents independently selected from halogen, —CH3, —OCH3, —CF3, and —OCF3.

26-29. (canceled)

30. The compound of claim 16, or a pharmaceutically acceptable salt thereof, which is selected from the compounds shown below:

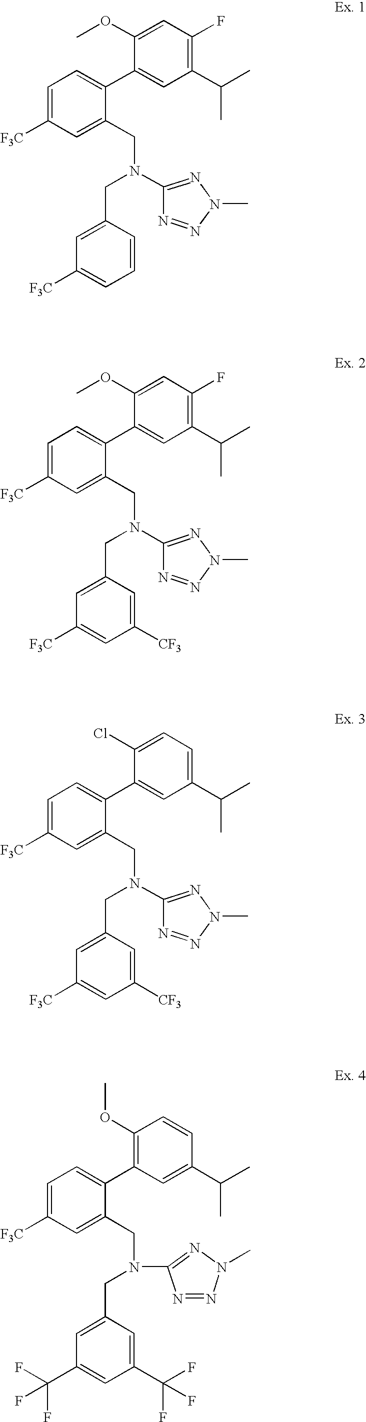

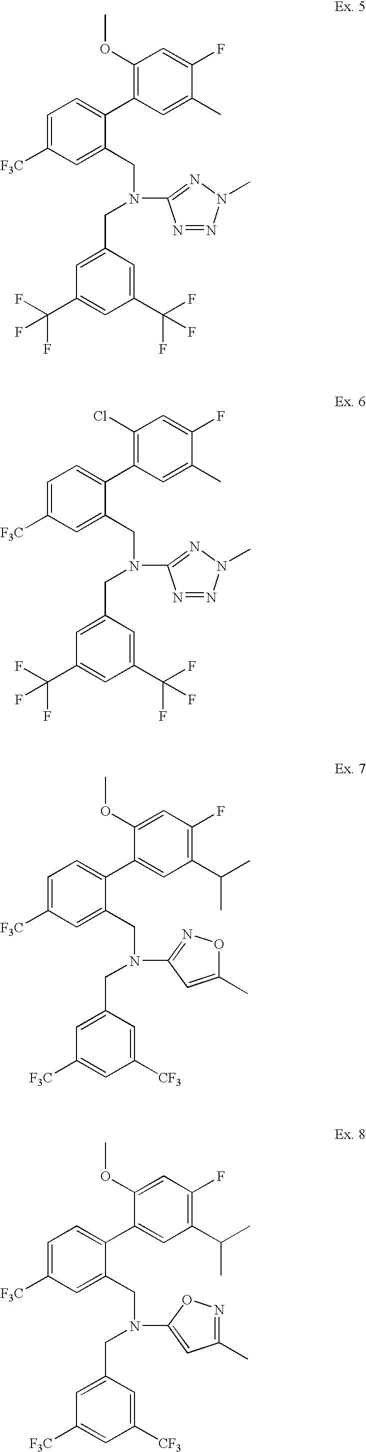

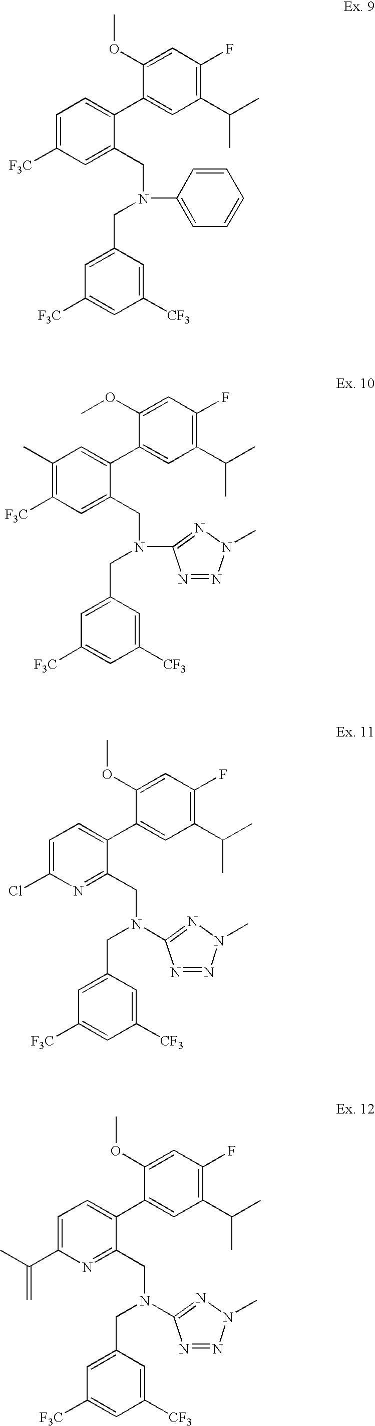

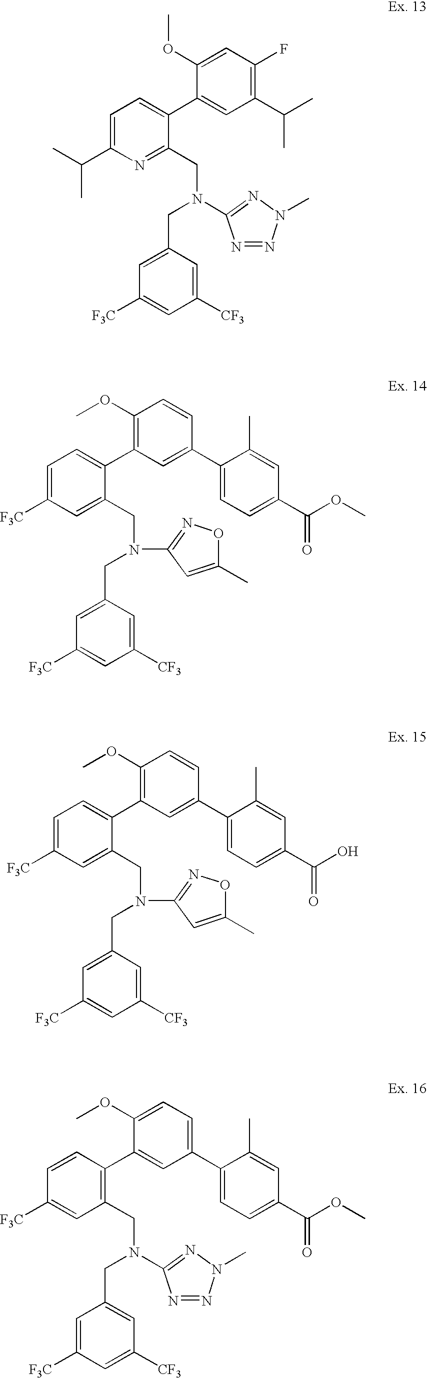

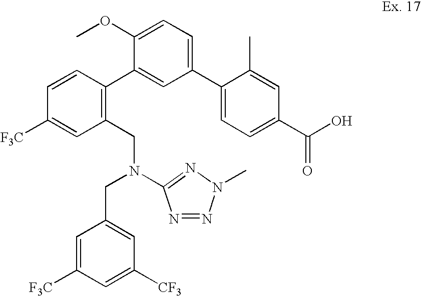

31. A method of treating atherosclerosis in a patient in need of treatment comprising the administration of a therapeutically effective amount of the compound of claim 1, or a pharmaceutically acceptable salt thereof, to said patient.

32. A method of raising HDL-C in a patient in need of treatment comprising the administration of a therapeutically effective amount of the compound of claim 1, or a pharmaceutically acceptable salt thereof, to said patient.

33. (canceled)

34. A pharmaceutical composition comprising the compound of claim 1, or a pharmaceutically acceptable salt thereof, and a pharmaceutically acceptable carrier.

35. A pharmaceutical composition comprising the compound of claim 1, or a pharmaceutically acceptable salt thereof, and one or more active ingredients selected from the group consisting of:
(a) PPAR gamma agonists and partial agonists;
(b) biguanides;
(c) protein tyrosine phosphatase-1B (PTP-1B) inhibitors,
(d) dipeptidyl peptidase IV (DP-IV) inhibitors;
(e) insulin or insulin mimetics;
(f) sulfonylureas;
(g) α-glucosidase inhibitors;
(h) one or more compounds selected from the group consisting of (a) HMG-CoA reductase inhibitors; (b) bile acid sequestrants; (c) niacin, nicotinyl alcohol, nicotinamide, and nicotinic acid or a salt thereof; (d) PPARα agonists; (e) cholesterol absorption inhibitors; (f) acyl CoA:cholesterol acyltransferase (ACAT) inhibitors; (g) phenolic anti-oxidants, such as probucol, and (h) a microsomal triglyceride transfer protein (MTP)/ApoB secretion inhibitor;
(i) PPARα/γdual agonists;
(j) PPARδ agonists;
(k) antiobesity compounds
(l) ileal bile acid transporter inhibitors;
(m) anti-inflammatory agents;
(n) glucagon receptor antagonists;
(o) GLP-1,
(p) GIP-1,
(q) GLP-1 analogs;
(r) glucokinase activators; and
(s) antihypertensive compounds.
